(19) United States
(12) Patent Application Publication
(10) Pub. No.: US 2025/0288705 A1
(43) Pub. Date: Sep. 18, 2025

(54) APPARATUSES, SYSTEMS, AND METHODS FOR GROWTH TRAY STERILIZATION

(71) Applicant: Element Farms, Inc., Pompton Plains, NJ (US)

(72) Inventors: Daniel Janeczko, Newton, NJ (US); Pete Vegas, Boulder, CO (US); Serdar Mizrakci, New York, NY (US)

(73) Assignee: Element Farms, Inc., Pompton Plains, NJ (US)

(21) Appl. No.: 18/931,971

(22) Filed: Oct. 30, 2024

Related U.S. Application Data
(63) Continuation-in-part of application No. 18/502,692, filed on Nov. 6, 2023.

(51) Int. Cl. A61L 2/04; A61L 2/18; A61L 101/32

(52) U.S. Cl. CPC A61L 2/04; A61L 2/18; A61L 2101/32; A61L 2202/23

(57) ABSTRACT

Methods and apparatuses for sterilizing growth trays used to hydroponically grow crops. The growth trays comprise a plurality of openings for holding soil and germinating seeds in the soil. The plurality of openings having a profile that allow the roots of the growing plant to extend into the bottom tub. The methods comprising applying at least one chemical cleaner to trays, drying the trays to a desired percent moisture, and heating the trays using a dielectric apparatus to kill at least one pathogen to produce sterilized growth trays. A method of hydroponically growing crops in the sterilized growth trays.

This application claims the benefit of priority as a Continuation in Part of U.S. Utility application Ser. No. 18/502,692, filed Nov. 6, 2023, which is incorporated herein by reference in its entirety.

### TECHNICAL FIELD

The present disclosure is in the field of Agriculture. This application relates to a hydroponic method for rapidly growing a vegetable crop. More particularly, this application relates to hydroculture, particularly to apparatuses, systems, and methods for the sterilization of growth trays and other materials used in hydroponics.

### BACKGROUND

Hydroculture and its subset hydroponics have been a rapidly growing system for growing corps using mineral nutrient solutions in water as a solvent. It is typically done in a green house or similar structure to provide a controlled environment. It can be done anywhere in the country and at any time during the year.

However, there is still room for substantial improvement in the technology. These improvements include use of light, design of the flats that hold the plants, and other improvements to eliminate harmful pests, bacteria and fungi that can cause extensive damage in a hydroculture operation.

In the last two decades, lettuce and basil production have increasingly moved indoors from fields to Controlled-Environment Agriculture (CEA) facilities like greenhouses or warehouse-style vertical farms. A primary reason for this trend is that lettuce and basil plants are well-suited to the commercial hydroponic systems on the market today in terms of growth habit and with the exception that they are susceptible to certain plant pathogens. Vertical farms along with greenhouses taking advantage of sunlight using systems such as the Nutrient Film Technique (NFT) and Deep-Water Culture (DWC) systems provide space-efficient growing environments for skilled operators who strive to make a margin on these crops in premium markets. With growing systems from the likes of Green Automation, Prims, and American Hydroponics for NFT systems or Dry Hydroponics, Hydronov, or Viscon system for DWC, many hectares of production have been added in North America with this trend accelerating as weather changes in the West. Spinach on the other hand, although it's consumed in similar quantities in the US, is produced by very few CEA facilities. The main reason for this is that spinach is much more susceptible to root pathogens that other leafy greens.

Once an infection by root pathogens starts in a facility, it has the capacity to infect all portions of the growing facility including plumbing, trays, equipment, etc. and decimate crops rapidly. Infection by the oomycete pathogen Pythium is a common cause of “damping-off” in spinach and other crops, which is plant pathology nomenclature for having an early root infection just after germination that can stunt growth and even kill the plant. Even when infection is not fatal to plants, Pythium can severely reduce yields and affect shelf-life of harvested product.

Pythium is a genus of oomycete organisms with some strains being aggressive root pathogens of spinach, as well as other commonly grown hydroponic crops including cucumbers, lettuce, arugula, and cannabis. Pythium belongs to Kingdom Chromista, neither animal, plant, nor true fungus. Moreover, it has a unique life cycle. Pythium is ubiquitous and can be introduced into hydroponic systems by seeds, growing substrate, insect pests, employees, and even by the wind.

Spinach has relatively high susceptibility to pythium in the field, and especially in hydroponic systems which provide an ideal environment for pythium to proliferate if unchecked. It's been suggested that spinach's vulnerability to pythium in hydroponics could be partially due to Spinach producing an especially large amount of root exudates which may serve as a chemotaxic signal to swimming pythium zoospores.

There has been significant research on pythium in hydroponic production, including spinach. Many solutions have been tested and there are countless products on the market today that claim to reduce pythium infection. Some of these solutions include supersaturation of Nutrient Solution (NS) with nanobubbles or ozone, water filtration and various methods of water disinfection including ozone, oxidizing chemicals, and UV lights. In field production it is common to use systemic fungicides in irrigation water or as a coating for seeds. These pesticides are not labeled for hydroponic production. Other solutions that have been tested include quarantining crops to prevent backflow of pathogens, beneficial microbial inoculants, aeroponic production, and sonication/pasteurization of the NS to name a few.

There are a few ways to reduce the presence of water-borne pathogens in hydroponics. Water quality can be monitored, filtered, and sterilized to remove of kill pathogens. This may occur before, during, or after plants are placed into a hydroponic growth media, such as a pond.

Although this is an effective form of preventing pathogen grown in hydroponic systems, it does not guarantee that all of the pathogens will not be introduced into the pond. Pathogens can infect a system through accidental human introduction or contaminated growth materials, for example. The trays or flats that the crops grow in can, for example, contain trace amounts of pathogens that then reproduce and infect a pond. It is not cost efficient, or environmentally friendly, to purchase/use single-use trays. Tray sterilization is typically done using chemicals. However, because trays are typically porous and made out of material, e.g. Styrofoam, that floats on water, pathogens can hide away in the pores of the material and escape the reach of sterilization. When the trays are reused then the pathogen blooms in the water, infects the pond, and harms the crop.

The disclosed tubs, systems, and methods to sterilize trays more effectively are directed to overcoming one or more of the problems set forth above and/or other problems of the prior art.

### SUMMARY

Consistent with some embodiments, methods of sterilizing growth trays to kill pathogens are disclosed. In some embodiments, the methods comprise washing growth trays with water at least once; applying at least one chemical cleaner to the trays; drying the trays to a desired percent moisture, such as less than 30% moisture by weight; and heating the trays using a dielectric apparatus that kills at least one pathogen, such as a microwave.

In some embodiments, washing is performed using a power or high-pressure washer.

In some embodiments, applying at least one chemical cleaner is performed while also subjecting the growth trays to ultrasonic energy.

In some embodiments, applying at least one chemical cleaner comprises: rinsing the trays with a quaternary ammonium sanitizer spray, and then submerging the trays in a concentration of up to 300 ppm quaternary ammonium solution for less than 1 minute.

In some embodiments, drying the trays may be performed with at least one fan and is used to achieve a moisture content ranging from 5 to 15%.

In some embodiments, the dielectric apparatus comprises a conveyor belt, a drying tunnel, a controlling system; wherein the conveyor belt is located inside the drying tunnel and the controlling system is attached to the drying tunnel; wherein the drying tunnel has emitters, such as at least four emitters that have a microwave power of approximately 10 kW and an output frequency of approximately 2450 MHz.

### BRIEF DESCRIPTION OF THE DRAWINGS

The accompanying drawings, which are incorporated in and constitute a part of this specification, illustrate some disclosed embodiments and, together with the description, serve to explain the disclosed embodiments. The particulars shown are by way of example and for purposes of illustrative discussion of embodiments of the present disclosure. The description taken with the drawings makes apparent to those skilled in the art how embodiments of the present disclosure may be practiced.

FIG. 1A is a top view of a growth tray with oval openings consistent with some disclosed embodiments.

FIG. 1B is a schematic sectional view (section A-A of FIG. 1A) of the openings consistent with some disclosed embodiments.

FIG. 1C is an exploded view of section B in FIG. 1B showing an opening consistent with some disclosed embodiments.

FIG. 2A is a top view of a tub used in a fully-quarantine mobile tub system consistent with some disclosed embodiments.

FIGS. 2B and 2C show the side view and end view, respectively, of the tub in FIG. 2A.

FIGS. 3A and 3B are top and bottom perspectives, respectively, of a tub consistent with some disclosed embodiments.

FIG. 4A is a side perspective of a tub with the inlet section for an air bubbler highlighted as Detail A, consistent with some disclosed embodiments.

FIG. 4B is an exploded view of Detail A from FIG. 4A.

FIG. 5A is a side perspective of a tub with at least one corner of the top portion that includes a structure for keeping growth trays from floating off the tub when the tub is filled with said aqueous mixture highlighted as Detail B, consistent with some disclosed embodiments.

FIG. 5B is an exploded view of Detail B from FIG. 5A.

FIG. 6A is an end perspective of an unfilled tub with the growth tray sitting on the top lip of the tub consistent with some disclosed embodiments.

FIG. 6B is a cross-sectional view (section A-A of FIG. 6A) showing how the growth tray sits on the top lip of the unfilled tub.

FIG. 7A is an end perspective of a filled tub with the growth tray sitting on the top lip of the tub consistent with some disclosed embodiments.

FIG. 7B is a cross-sectional view (section A-A of FIG. 7A) showing how the growth tray floats above the upper lip of the tub because of the aqueous nutrient solution that is in the filled tub.

FIG. 8A is a side perspective of an unfilled tub with the growth tray sitting on the top lip of the tub consistent with some disclosed embodiments.

FIG. 8B is a side perspective showing how the growth tray floats above the upper lip of the tub because of the aqueous nutrient solution that is in the filled tub consistent with some disclosed embodiments.

FIG. 8C is an exploded view of top corner of the tub shown Detail A of FIG. 8B consistent with some disclosed embodiments.

FIGS. 9A, 9B and 9C are schematics showing: a top perspective OF the growth trays (FIG. 9A), a side perspective of growth trays on top of the tubs (FIG. 9B), and a bottom perspective of the tubs (FIG. 9C), all on a rail system consistent with some disclosed embodiments.

FIG. 10A illustrates a rail system for moving multiple fully-quarantine mobile tubs according to an embodiment consistent with the present disclosure. FIG. 10B illustrates a side perspective of one fully-quarantine mobile tub shown in FIG. 10A.

FIG. 11A illustrates a rail system for moving multiple fully-quarantine mobile tubs according to an embodiment consistent with the present disclosure. FIG. 11B is an exploded view of a connection member shown in FIG. 11A that is consistent with embodiments of the present disclosure. FIG. 11C is an exploded view of roller members shown in FIG. 11A that is consistent with embodiments of the present disclosure.

FIG. 12 is a flow diagram of method of growing plants using a fully-quarantine mobile tub consistent with some disclosed embodiments.

FIG. 13 is a flow diagram of a prior art method of growing plants in a pond system.

FIG. 14 is an example of an apparatus or part of an apparatus used to wash trays consistent with some disclosed embodiments.

FIG. 15 is a side view of an example of the trays being dried using box fans consistent with some disclosed embodiments.

FIG. 16 is an angled view of a dielectric apparatus consistent with some disclosed embodiments.

FIG. 17 is a side view of a dielectric apparatus consistent with some disclosed embodiments.

FIG. 18 illustrates a tub system according to an embodiment consistent with the present disclosure with nascent plants and a top raft floating on top of the water below.

### DETAILED DESCRIPTION OF INVENTION

The following disclosure generally describes components, systems and methods to significantly reduce infection by pythium and other root pathogens in Deep Water Culture (DWC) spinach production. In one embodiment, there is described a container for hydroponically growing plants, comprising: a top tray that floats in water configured to sit on a bottom tub containing water and nutrients. The top tray described herein comprises a plurality of openings completely therethrough for holding soil and germinating seeds in the soil and water, the plurality of openings having a profile that allow the roots of the growing plant to extend into the bottom tub as the roots search for water and nutrients. In an embodiment, the tub is configured to hold a mixture of water and nutrient solution for the growing plants, the tub comprising a lip around the top circumference and at least one air diffuser for causing a turbulent flow of the water mixture that is contact with the roots of the growing plants.

The top tray and the tub are configured to allow the top tray to float on the water in the tub for a few days while the young roots of the germinating seeds need full immersion in water. As the plants and evaporation consumes water the floating tray drops into the tub until it seats on a ledge. Once seated on ledge the tray in now in a position that will allow the plants to be commercially harvested in a typical cutting system, that cuts the plants much like a hedge trimmer. As the water level continues to drop, the roots continue to grow in length in search of water and nutrients causing the upper layers of the roots to be exposed to air. The roots that are in contact with the water are exposed to a very aggressive flow of bubbles generated by the bubblers. Success has been achieved by maintaining a water level in the tub, but it is not necessary and does not provide the advantages of allowing the water level to drop and roots to be exposed to air.

In particular, the disclosed tubs, systems, and methods allow roots of growing plants to continue to grow into the water by taking advantage of a dropping water level, that leads to several advantages. These advantages include (a) keeping the bursting bubbles and turbulence closer to the root tips as they continue to grow; (b) introducing an air gap that allows mature roots to have maximum access to fresh air and oxygen and less exposure to pathogens while still being able to get nutrients and water from their tips; having less weight to move and reduced splashing at harvest since the water level drops; having less water to filter after the trays are harvested; and resulting in a drier substrate which reduces pest, plant, and human pathogen growth close to the stems of plants.

In some embodiments, the tubs are relatively small, typically less than 20 square feet, or even less than 15 square feet, and the water and nutrients in the tubs are completely isolated from other tubs and water handling systems to prevent cross contamination of pathogens. The tubs can easily be completely sterilized as often as each growth cycle if needed. The tubs are designed to be mechanically combined in a system that can be mechanized and efficiently operated at large scale such as a 10-acre greenhouse.

Consistent with disclosed embodiments, there is also described a method of hydroponically growing plants. In an embodiment, the method comprises planting a plurality of seeds in growth media, where the growth media are contained in plurality of trays that are configured to float on top of a tub and to provide access for the crops to the water mixture when floating. As mentioned, the tub comprises a lip around the top circumference and containing a mixture of water and nutrient solution. The method further comprises germinating the seeds to produce trays with germinated seeds and transferring the trays with germinated seeds to the top of the tub. The trays are then floated on top of the tub until they are allowing the tray to drop closer to the tub as the water level in the tub decreases. As the water level decreasing as a result of being taken up by the growing plants, the tray moves down to sit on the lip of the tub such that a portion of the roots remained exposed to air and a portion of the roots in contact with the mixture of water and nutrient solution. The method comprises at least one aeration steps that causes a turbulent flow of the water mixture to impinge on the roots of the plant that are in contact with the mixture of water and nutrient solution.

There is described a fully quarantined, meaning that each tray is sitting in an isolated tub of Nutrient Solution (NS) which is typically less than 20 square feet, such as 15 square feet in size and yet can be combined with other tubs in such a way that a large greenhouse (10 acres as an example) can be fully mechanized and commercially efficient. In one embodiment, there is described herein methods of growing spinach, other leafy greens, or vegetables (all to be called “plants” for the purposes of this document) in very small batches where the water source is isolated from other batches so that disease cannot spread to their roots from other plants.

The isolation prevents the movement of water between the tubs and mitigates the transfer of plant pathogens. It may offer some other plant-health advantages in addition to labor saving and food-safety advantages; and (2) it employs unusually large aerators and high airflow capacity to constantly cover plant roots in a rising cascade of bubbles that wash away exudates and prevent Pythium spore germination while rapidly agitating the plant roots.

In some embodiments, the disclosed tubs, systems, and methods to sterilize trays more effectively are directed to overcoming one or more of the problems set forth above and/or other problems of the prior art. In particular, the disclosed tubs, systems, and methods allow roots of growing plants to continue to grow into the water by taking advantage of a dropping water level, that leads to several advantages. In some embodiments, these advantages include (a) keeping the bursting bubbles and turbulence closer to the root tips as they continue to grow; (b) introducing an air gap that allows mature roots to have maximum access to fresh air and oxygen while still being able to get nutrients and water from their tips; having less weight to move and reduced splashing at harvest since the water level drops; having less water to filter after the trays are harvested; and resulting in a drier substrate which reduces pest, plant, and human pathogen growth close to the stems of plants.

This concept of reducing infection by Pythium and other root pathogens through vigorous bubbling of the roots was developed through understanding how pythium infection occurs on a microbiological level in wet conditions. It has been discovered that germinating spores of pythium species release swimming agents of infection that attack plant roots. These motile zoospores are attracted to root exudates of young plants. Spinach produces an unusually large quantity of these exudates, which is part of the reason it is especially vulnerable to infection. To address this problem, the present disclosure provides for intense bubbling in the disclosed system. This mechanism serves to wash away root exudates from the root surface while their constant movement prevents the motile pythium zoospores from attaching on root tips to germinate. By preventing infection by root pathogens, vigor and yield of hydroponically grown spinach is increased substantially allowing for significantly higher yields (kg/m2/year) than offered by other growing systems with improved consistency.

In one embodiment, the fully quarantined mobile tub spinach system described herein employs an unusually large aeration capacity. For example, in one embodiment of the present disclosure, a spinach system disclosed herein targets air flow of 25 L/min through an 8 inch air diffuser in a 20 gal reservoir. Various diffusers may be used with the understanding that they have a high capacity for air flow. In some embodiments, the disclosed method comprises pumping large amounts of dissolved oxygen in order to bubble a nutrient solution specific for the leafy greens to be grown.

In an embodiment, the amount of aeration used in the disclosed process is larger than 3 L/min per 20 gal, with the dissolved oxygen being impact directly on the roots of each growing plant. This is not possible in current systems that use large growing ponds. In some embodiments, it has been discovered that bubbling their NS in the amounts and at the locations (right at the root surface) described herein causes vigorous aeration and prevents infection by root pathogens. Therefore, the disclosed systems and methods have shown an increase in yield of the harvested plant. It has been shown that high-intensity bubbling in the disclosed moving tub system is effective at mitigating and preventing infection by pythium spp. and other root pathogens in hydroponically grown plants, such as spinach.

In some embodiments, it has been demonstrated that even in the presence of pythium propagules in the NS, spinach can be grown healthily with the disclosed Tub Spinach System providing vigorous aeration. In one embodiment, it was shown that it was possible to grow high-yielding, healthy crop in the same water for several cycles without discharging it while only replacing the water the plants use to keep the reservoirs full.

More generally, in some embodiments the fully-quarantine Mobile Tub System (MTS) and method of using the same according to the present disclosure starts with seeds that are sown in described seedling trays. Seeding depth, water content of the soilless mix, germination temperature/duration, and soil compaction are controlled such that: (a) seed coats are removed from emerging seedlings at over 95%; (b) homogeneous emergence of seedlings; (c) Roots protrude from the bottoms of trays before planting into tub system. This ensures root tips, the most vulnerable part of the juvenile seedlings, is immersed in the bubbling cascade immediately and the root tip does not spend time in water-logged soil where it can be quickly infected. The Inventors have found that the success of the system starts with growth trays.

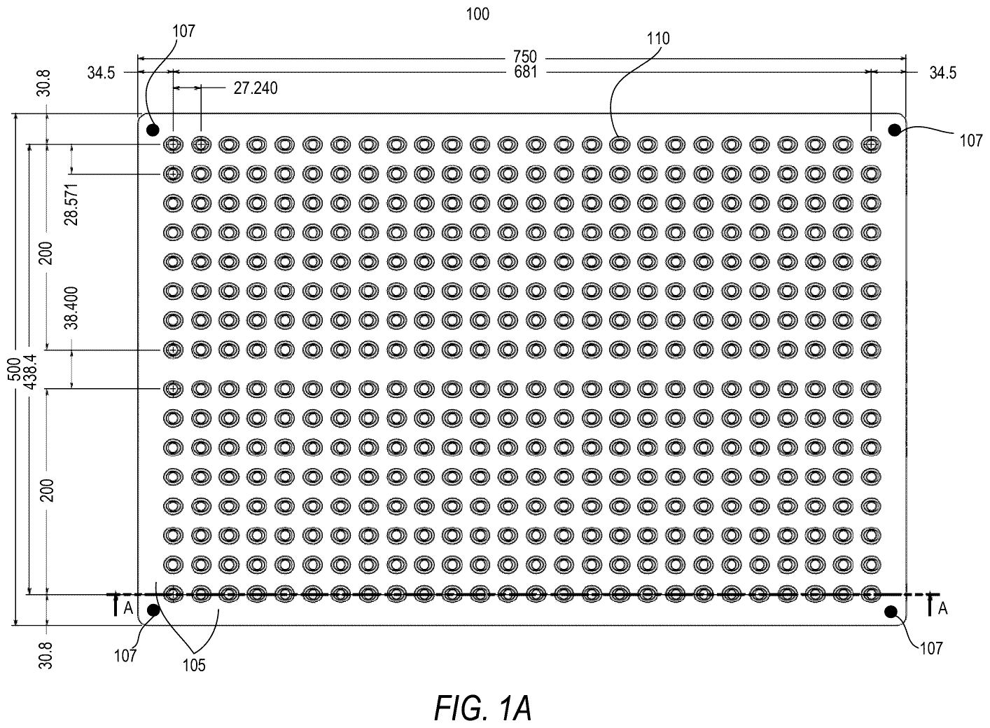

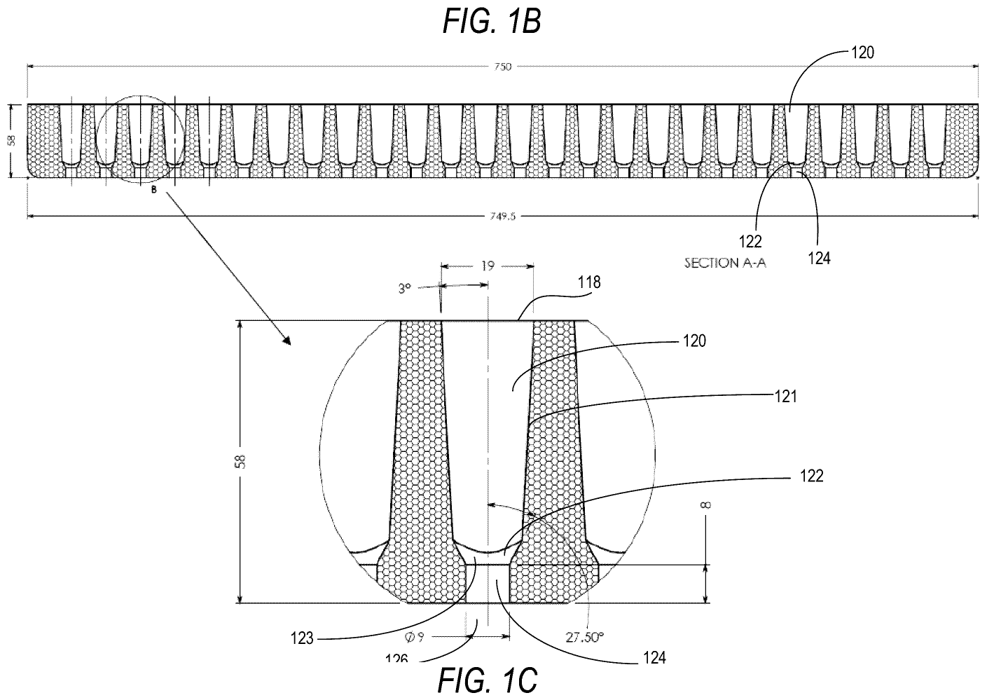

In some embodiments, there is described a growth tray configured to contain and germinate plants. The growth tray described herein is configured to sit and float on an aqueous solution that is contained in a tub, wherein the growth tray has a top surface and a bottom surface with a plurality of openings completely through the top surface and the bottom surface. FIG. 1A shows a top surface of a growth tray 100 having a plurality of openings 110. In some embodiments, the openings 110 have an oval shape.

In some embodiments, the growth trays have a rectangular shape that is 450-550 mm wide, such as 475-500 mm wide. In some embodiments, the growth trays are 700-800 mm long, such as 725-775 mm long. In some embodiments, the growth trays are 50-60 mm thick, such as 52-58 mm thick. In some embodiments, the growth trays have 400-450 openings, such as 410-430 openings, or even 415-420 opening, and can grow from 500-1200 plants, such as 600-800 plants.

In some embodiments, the plurality of openings 110 are configured to hold soil and germinate seeds in the soil. In some embodiments, each opening 110 has a volume ranging from 8-10 ml, such as 9 ml.

With reference to FIGS. 1B and 1C, in some embodiments, the plurality of openings 110 comprising at least three regions, including a top region 120 having a top opening 118 sufficient for sprouting plants to grow through the top surface and sidewalls 121 having a tapered shape to a more narrowed transition region 122. In some embodiments, the sidewalls of the top region 121 have a taper angle of 2-4 degrees relative to a vertical plane drawn through the center of the openings, such as 3 degrees. In some embodiments, the top region 120 has a concaved shape bottom 123 leading into the transition region.

The plurality of openings 110 further include a second region, which is a transition region having sidewalls that further taper to a more narrowed end region 122. In some embodiments, the sidewalls of the transition region have a taper angle of 26-28 degrees relative to a vertical plane drawn through the center of the openings.

The plurality of openings 110 further include a third region, which is an end region having straight sidewalls with a bottom opening for plant roots to grow through the bottom surface and contact the aqueous solution that is contained in a tub 124.

In some embodiments, the plurality of openings have an oval shape 118 on the top surface of the growth tray 100 and a round shape on the bottom surface 126. In some embodiments, the oval shape on the top surface 118 of growth tray 100 has as its longest axis a diameter of 18-20 mm, such as from 18.5-19.5 mm. In some embodiments, the round shape 126 on the bottom surface of growth tray 100 has a diameter of 8-10 mm.

In some embodiments, the top surface of growth trays 100 includes a border around the edge that is free of openings 105. In some embodiments, the border 105 has a size at least as wide as the longest axis of the oval shaped openings 118.

In some embodiments, the top surface of growth trays 100 includes a plurality of tabs 107 that are located on the border 105. The plurality of tabs 107 are configured to allow growth trays to be stacked on each other without the bottom surface of a top growth tray touching the top surface of a bottom growth tray stacked on top of it.

In some embodiments, there is described a growth tray configured to contain and germinate plants. In some embodiments, the growth tray comprises expanded polystyrene (EPS) and configured to sit and float on an aqueous solution that is contained in a tub.

In some embodiments, the growth tray has a top surface and a bottom surface with 400 to 450 oval-shaped openings completely through the top surface and ending at a circular shaped opening at the bottom surface. In some embodiments, the oval-shaped openings are configured to hold soil and germinate seeds in the soil, and comprise at least three regions: a top region, a transition region and a bottom region.

In some embodiments, the top region has top, oval-shaped openings having as its longest axis a diameter of 18-20 mm and tapered sidewalls having a taper angle of 2 to 4 degrees relative to a vertical plane drawn through the center of the openings, wherein the top region has a concaved shape bottom leading into a transition region.

In some embodiments, the transition region with tapered sidewalls having a taper angle of 26 to 28 degrees relative to a vertical plane drawn through the center of the openings that lead to a more narrowed end region.

In some embodiments, the end region has straight sidewalls with a bottom opening having a round shape and a diameter of 8-10 mm that allow plant roots to extend down through the bottom surface and contact the aqueous solution that is contained in a tub.

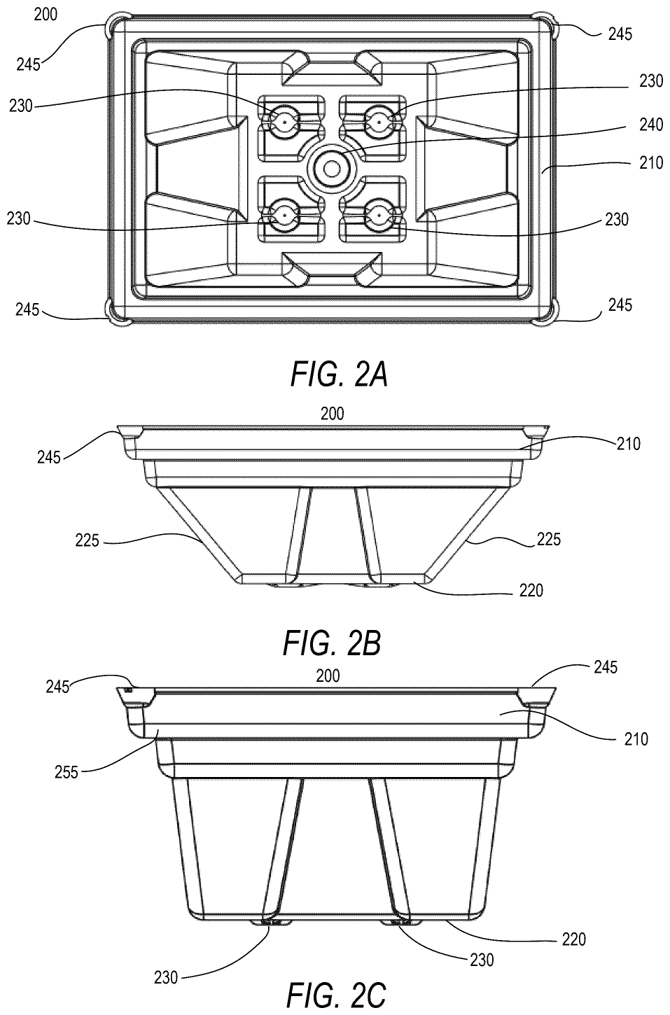

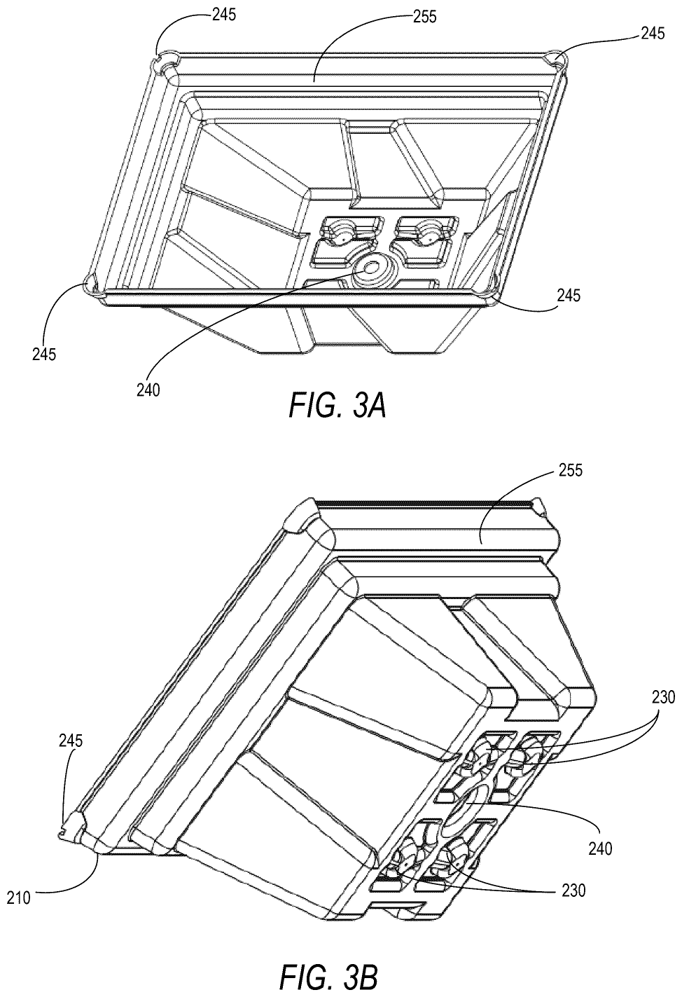

With reference to FIGS. 2A-2C and FIGS. 3A-3B, in some embodiments, there is described a tub 200 configured to have a growth tray to float thereon and sit therein and hold an aqueous mixture of water and a nutrient solution for growing plants. In some embodiments, the tub includes a top portion having a similar or same rectangular shape as the growth tray, and a lip around the internal circumference that is configured to receive the growth tray 210.

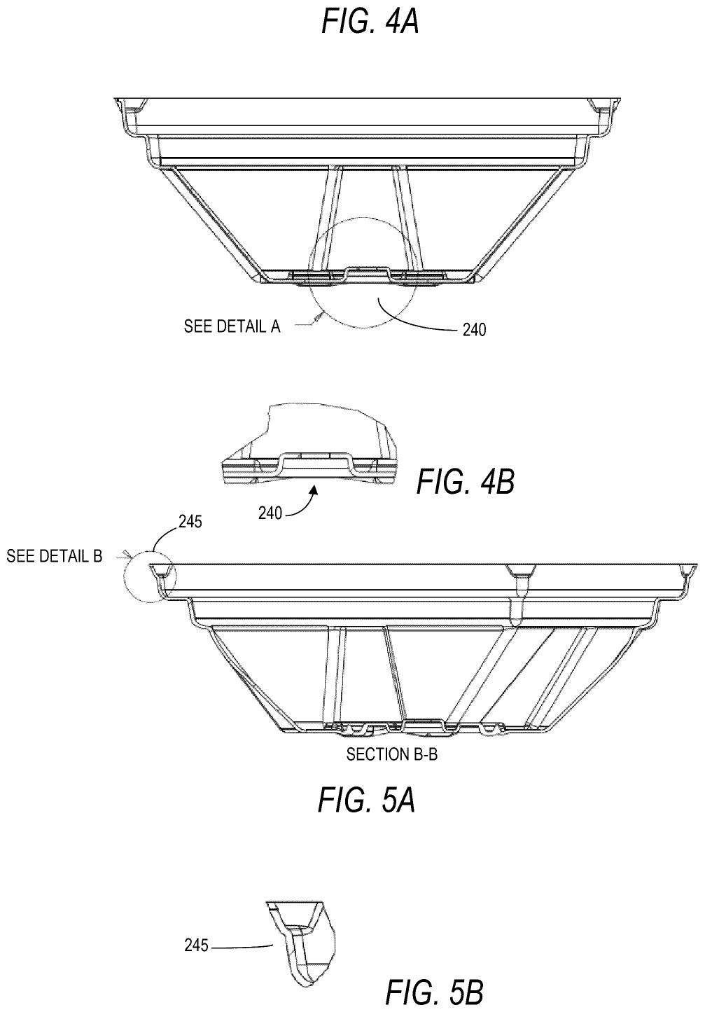

In some embodiments, the tub includes a bottom closed portion 220 having a rectangular shape smaller in size than the top portion 210. The bottom closed portion 220 may comprise at least one attachment mechanism 230 for removably attaching the tub 200 to a frame. With reference to FIGS. 4A and 4B, the tub includes a bottom closed portion 220 and an air diffuser 240 configured to create a turbulent flow of the aqueous mixture located in the tub 200.

In some embodiments, the air diffuser 240 creates an amount of aeration larger than 3 L/min per 20 gal. For example, in some embodiments, the air diffuser creates an amount of aeration of 25 L/min through an 8-inch air diffuser in a 20 gal reservoir.

In some embodiments, the sidewalls of the tub taper 225 from the top portion 210 to the bottom closed portion 220, wherein the tapered sidewalls 225 are configured to allow the roots hanging from the growth tray to grow toward the center of the tub and be impacted by the turbulent flow of the aqueous mixture located in the tub 200. In some embodiments, a majority of the roots hanging from the growth tray that grow toward the center of the tub are contacted with the amount of aeration.

In some embodiments, the lip around the internal circumference of the top portion 210 is configured to receive a growth tray may have a thickness from 50-60 mm. In some embodiments, the lip around the internal circumference of the top portion 210 includes a ledge 255 for resting the growth tray when the amount of aqueous mixture in the tub is not enough to float the growth tray.

With reference to FIGS. 5A and 5B, in some embodiments, the lip around the top portion 210 includes on at least one corner of the top portion a structure 245 for keeping growth trays from floating off the tub when the tub is filled with said aqueous mixture. The structure 245 may further includes an opening for removing excess aqueous mixture from the tub.

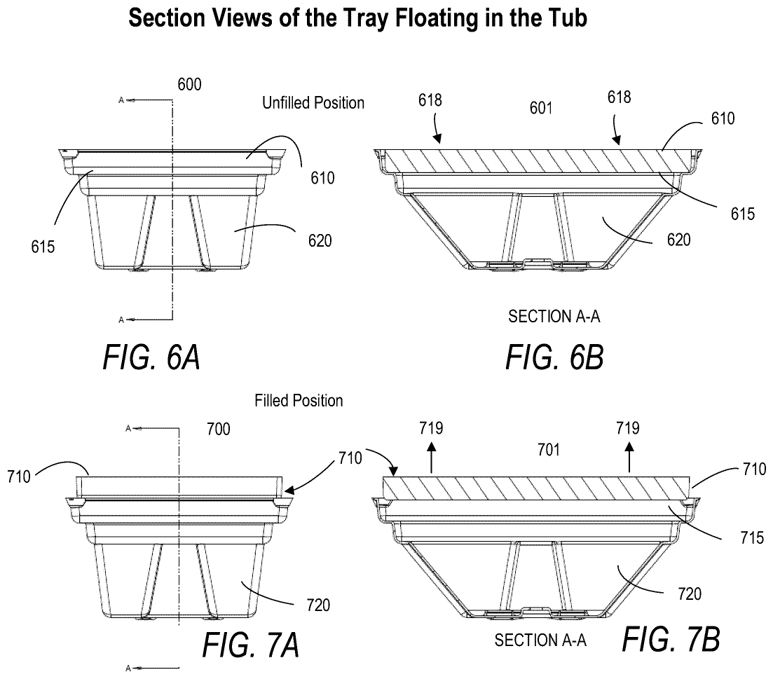

FIGS. 6A and 6B are sectional views of growth tray 610 sitting down (represented by down arrows 618) on the internal lip 615 of an unfilled tub 620. FIG. 6A is a side perspective 600 and FIG. 6B is a cross-sectional perspective 601 through the A-A plane shown in FIG. 6A.

In contrast to the unfilled tub 620 shown in FIGS. 6A and 6B, FIGS. 7A and 7B show the location of the floating seed 710 when tub 720 is filled with a nutrient rich solution. FIG. 7A is a side perspective 700 and FIG. 7B is a cross-sectional view 701 (section A-A of FIG. 7A) showing how the growth tray 710 floats above the upper lip of the tub (represented by up arrows 719) because of the aqueous nutrient solution that is in the filled tub.

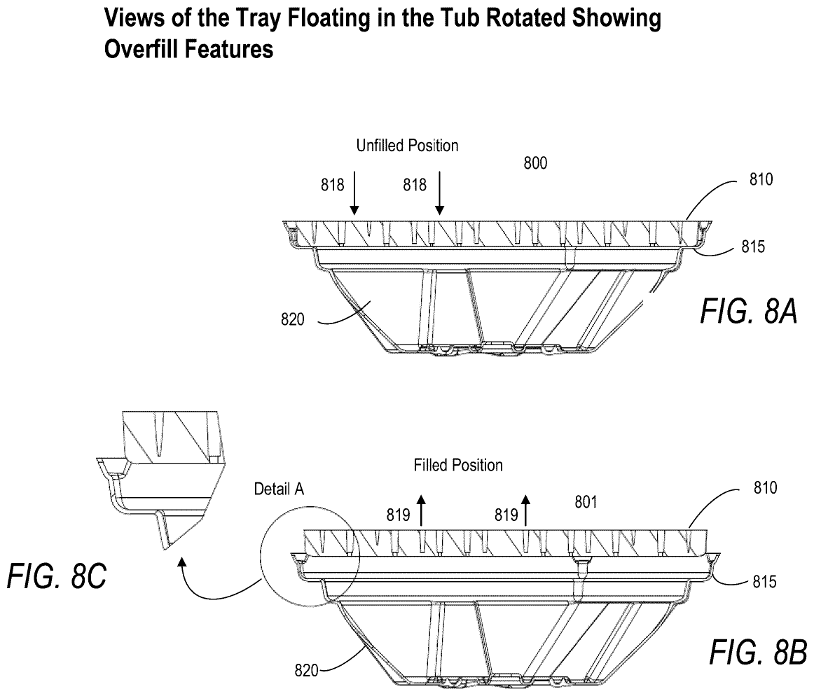

FIGS. 8A and 8B are similar to FIGS. 6B and 7B, respectively. In particular, FIG. 8A, like FIG. 6B, is a cross-sectional view of an unfilled tub 820 with growth tray 810 sitting on the top lip 815 of tub 820. FIG. 8B is a side perspective showing how the growth tray floats above the upper lip of the tub because of the aqueous nutrient solution that is in the filled tub consistent with some disclosed embodiments. In particular, like FIG. 7B, is a cross-sectional view of an filled tub 820 with growth tray 810 floating above the top lip 815 of tub 820, as shown by arrows 819.

FIG. 8C is an exploded view of top corner of the tub shown Detail A of FIG. 8B, which is described as an overflow feature to allow access water and nutrient rich solution to leave tub 820.

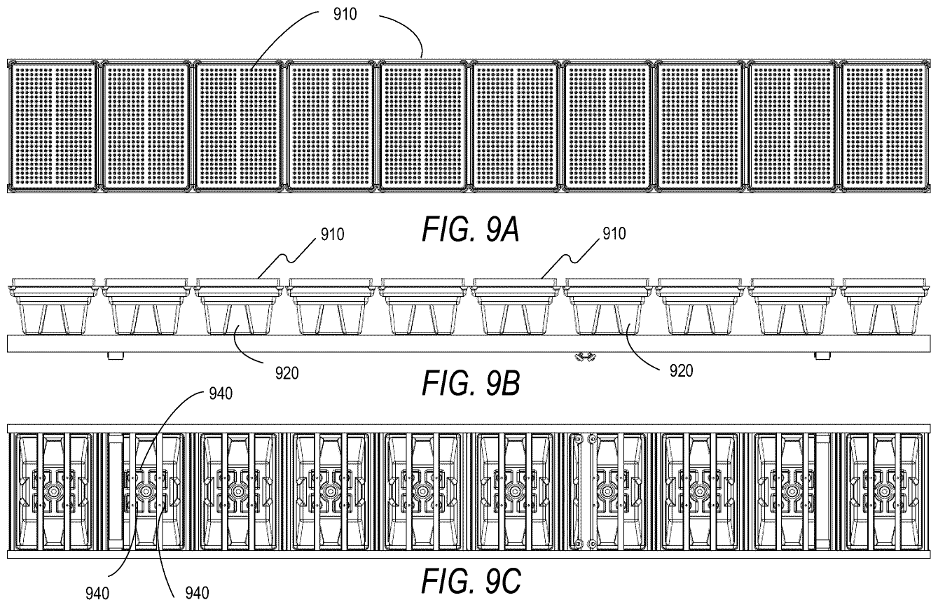

With reference to FIGS. 9A, 9B and 9C there is shown different perspectives of a rail system consistent with some disclosed embodiments. For example, FIG. 9A shows a top perspective of growth trays 910 lined-up side-by-side. FIG. 9B shows a side perspective of with growth trays 910 on top of the tubs 920. Finally, FIG. 9C show a bottom perspective of the tubs 920 connected to rails 930 by at least one attachment mechanism 940.

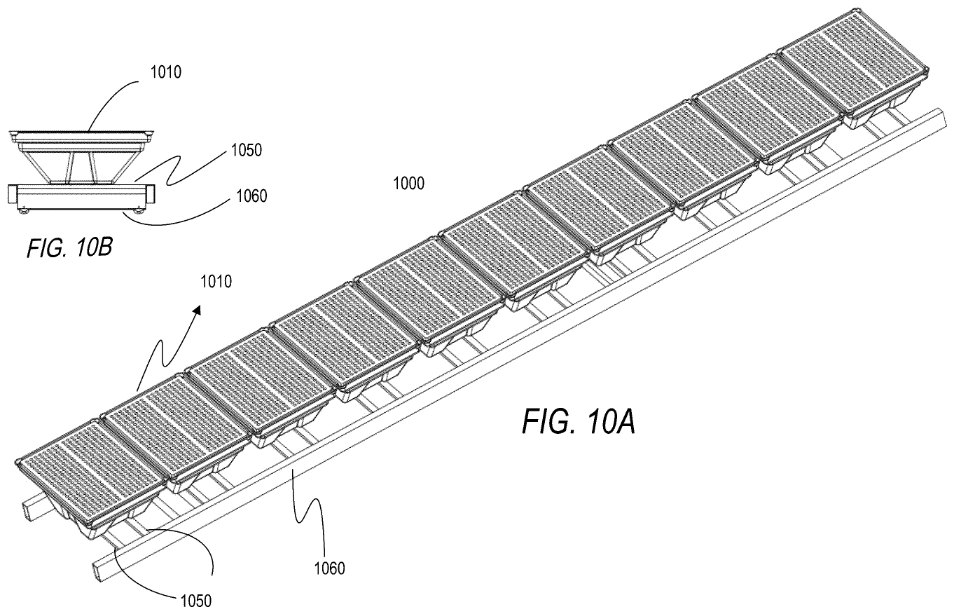

FIG. 10A illustrates a different perspective of the rail system shown in FIGS. 9A-9C for moving multiple fully-quarantine mobile tubs according to an embodiment consistent with the present disclosure. For example, FIG. 10A shows tubs 1010 removably attached to members 1050, which are perpendicular to rail members 1060. FIG. 10B further illustrates a side perspective of one fully-quarantine mobile tub shown in FIG. 10A.

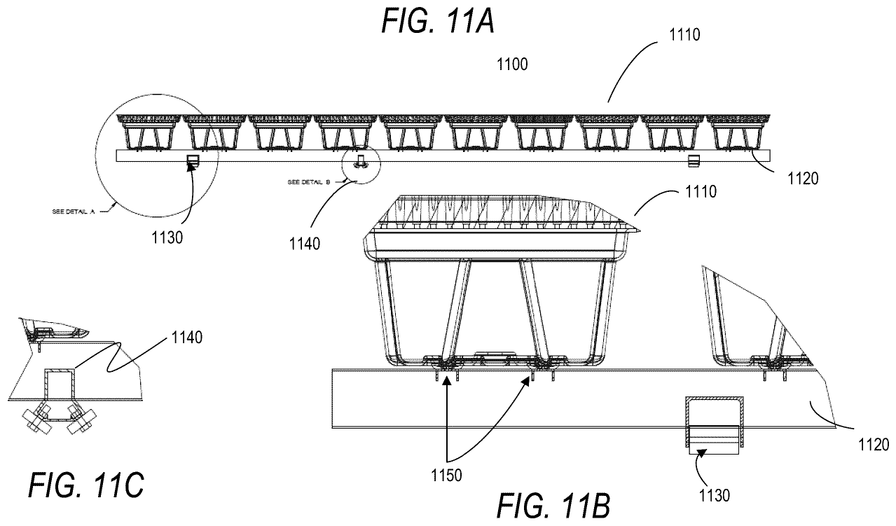

FIG. 11A illustrates a rail system for moving multiple fully-quarantine mobile tubs according to an embodiment consistent with the present disclosure. FIG. 11A illustrates a side perspective of the rail system for moving multiple fully-quarantine mobile tubs 1100 on rail members 1120 and rollers 1130 and 1140, that allow the multiple fully-quarantine mobile tubs 1100 to move in multiple directions. FIG. 11B is an exploded view of the connection members 1150 that removably secure the tubs 1110 to rail member 1120, which further includes roller 1130. FIG. 11C is an exploded view of roller member 1140. In some embodiments, the combination of rollers 1130 and 1140 allows the tubs to be moved in an X, Y direction, such as 90 degrees from each other.

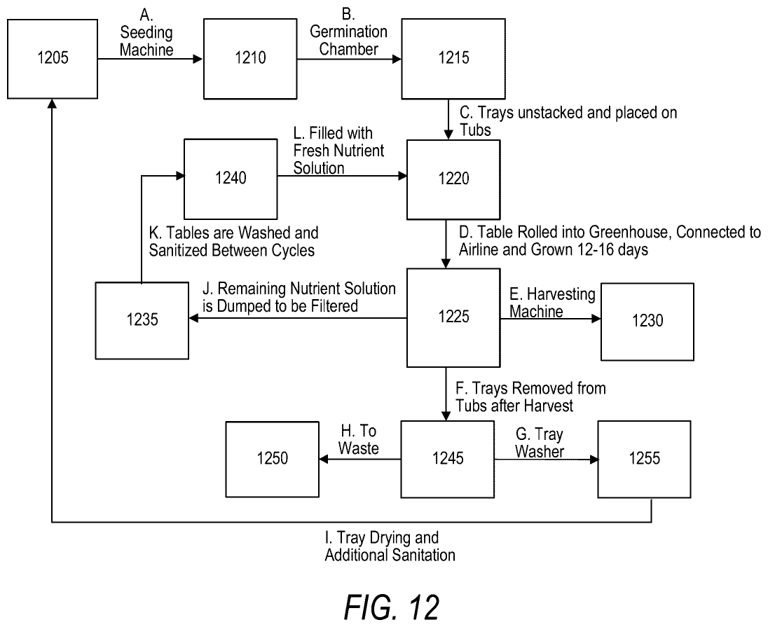

Consistent with the present disclosure, and with reference to FIG. 12, there is described a hydroponic growing system 1200. In some embodiments, the hydroponic growth system includes a method of cultivating plants without soil, using a nutrient-rich, aqueous-based solution to deliver essential minerals and nutrients directly to the plant roots.

In some embodiments, the system is based on a process that typically starts with a cleaned and sanitized growth trays 1205 that are used in seeding. Step (A) In some embodiments, a seeding machine may be used to automate the process of planting seeds or seedlings in step (A). Non-limiting embodiments of the growth trays that can be used in the disclosed system are more fully shown and described in FIGS. 1A-1C.

As described herein, seeds are typically sown in trays, flats, or seedling beds filled with a growing medium, such as nutrient-rich soil. In some embodiments, growth trays for hydroponics can be made of durable and food-safe materials, such as plastic or Styrofoam. These materials are lightweight, easy to clean, and resistant to water and nutrient solutions. In some embodiments, the growth trays come in various sizes and shapes, and are typically rectangular or square.

In some embodiments, as shown in FIGS. 1A-1C, growth trays are divided into multiple compartments or cells, each of which can hold one or more seeds. These compartments help keep seeds organized and prevent them from getting tangled or competing for resources as they grow. In some embodiments, the growth trays are made from Styrofoam or material capable of floating on water or liquid used in hydroponic systems. Trays may be made of other materials, such as plastics, organic composite matters, metals, and/or combinations thereof, provided that the material is able to float on top of a pond filled with liquid. In some embodiments, the growth trays may be, for example, expanded polystyrene (EPS) foam plug trays or flats. In some embodiments a separate plastic jacket can first be inserted into the cells of the flats or trays that are then filled with growth media.

The size and configuration of a particular tray will vary depending on the type of crop grown in the tray. For small plants naturally the cells will be closer together for larger plants they will be spaced further apart etc. The following particulars of sizes of trays are for a crop or cultivar of kale and is provided for illustrative purposes, other crops or cultivar would call for different configurations size but such changes would not depart from the concepts of the invention as those skilled in the art will appreciate once they understand to concepts of the invention and they would be able to adapt the tray to the particular crop without departing from the concepts of the invention. In the example of tray may be 25½ inches on its longitudinal axis 12½ on it short or latitudinal axis, 2 17/64 inch thick. Each of the oval cells is 13/16th of inch on the major axis of the top of the oval cell and the minor axis of the oval at the top of cell is 11/16th of an inch. Trays can be made of expanded polystyrene or any other similar light weight formable and buoyant material. In the prior art the floating tray or was simply a matrix of cells that covered the entire tray.

After seeding, the seeds are germinated in Step (B) by placing the growth trays 1210 into a germination chamber. In some embodiments, the growth trays are stacked inside the germination chamber with two or more trays stacked on top of each other. To allow the trays to be stacked without suppressing or damaging germinating plants, each tray includes at least one protrusion configured to provide a space between trays when stacked. In some embodiments, each tray includes at multiple protrusions located around the edges of the growth trays, such as at the corners of the trays.

The germination chamber used in step B, also known as a seed germination chamber or seedling incubator, is a specialized environment designed to facilitate the germination of seeds and the early growth of seedlings in hydroponic and traditional agriculture systems. It provides controlled conditions such as temperature, humidity, and sometimes light to optimize the germination process.

In some embodiments, the germination chamber maintains a consistent and controlled temperature, typically in the range of 70-85° F. (21-29° C.), which is optimal for seed germination. This temperature control helps speed up the germination process and ensures uniformity. The germination chamber may also maintain high humidity to prevent desiccation of the seeds. This is often achieved using misting systems or humidifiers.

In some embodiments, the germination chamber may be equipped with adjustable lighting systems to provide a consistent light source for seedlings. Additionally or alternatively, germination chambers may be placed in a separate growth area with appropriate lighting once germination occurs.

Adequate air circulation prevents the buildup of humidity and to ensure that the air around the seeds remains fresh and oxygen-rich. Small fans or ventilation systems may be included.

After the germination step (B) is complete, resulting in growth trays comprising sprouted seedlings, the trays 1205 are removed from the germination chamber 1215 and floated on top of the tubs 1220. Step (C) Consistent with some embodiments, the tubs have been cleaned and sanitized, and filled with a nutrient rich solution.

In some embodiments, the tubs 1220 with the sprouted seedlings trays 1215 may be placed on a table Step D. In some embodiments, the table contains one or more ports for receiving compressed air. In some embodiments, the one or more ports may be connected directly to the bottom of the tub to ensure a turbulent flow of are impinges the roots of the growing plant. In some embodiments, the one or more ports are connected to a hub that allows the compressed air to be distributed to multiple tubs simultaneously.

In some embodiments, as shown in step D, the table may be movable to allow it to be relocated, such as into or around a greenhouse. For example, in one embodiment the table may be moved, such as rolled, into a greenhouse where the plants will grow to maturity, typically in 11 to 16 days for spinach, such as 12 to 15 days, or 12 to 14 days. Step (D) In some embodiments, the tubs themselves may be configured to move independently of the other tubs.

To assist with seedling development and promote vegetative growth, lighting and environmental conditions may be adjusted, which typically includes more hours of light per day. In addition, the nutrient solution is monitored and may be adjusted to provide essential macro and micronutrients.

Once the plant has grown to maturity, it is ready for the harvesting 1225. Step (E). In some embodiments, harvesting is performed using a harvest machine. These machines are particularly valuable in large-scale commercial hydroponic operations where efficiency, speed, and precision are essential. In some embodiments, harvest machines are equipped with mechanisms to efficiently and gently handle the harvested crops. These mechanisms can include conveyor belts, robotic arms, or cutting blades, depending on the crop. One of the primary benefits of harvest machines is their ability to significantly increase the speed and efficiency of the harvesting process. They can harvest large quantities of crops in a short amount of time, reducing labor costs and increasing productivity. Another benefit of the disclosed system is the ability to move the entire table, with the mature plant contained in the disclosed tubs, through a harvest machine without having to remove the plant from the tub, or the tub from the table. This allows the harvesting step to be both efficient and economical.

After harvesting, the harvested plant is moved to cold storage 1230. The growth trays are then removed from the tub. Step F. Any remaining Nutrient Solution is removed from the tub. Step J. In some embodiments, the used Nutrient Solution may be filtered and re-used. For example, in some embodiments, the Nutrient Solution is filtered using basic mechanical filtering, such as with a 5 micron filter to eliminate spores. In some embodiments, the filtered Nutrient Solution may then be further cleaned such as with one or more methods including chemical sterilization, ozone treatment, or UV radiation.

In some embodiments, both the tubs and the growth trays are thoroughly cleaned and sterilized in preparation of the next growth cycle. For example, once the remaining NS is dumped to be filtered (Step J), the dirty tub 1235 is cleaned and sanitized, as are the tables and any other elements used during the growth cycle. Step K, resulting in cleaned and sanitized tubs 1240 to be filled with fresh NS and reused.

### Step L.

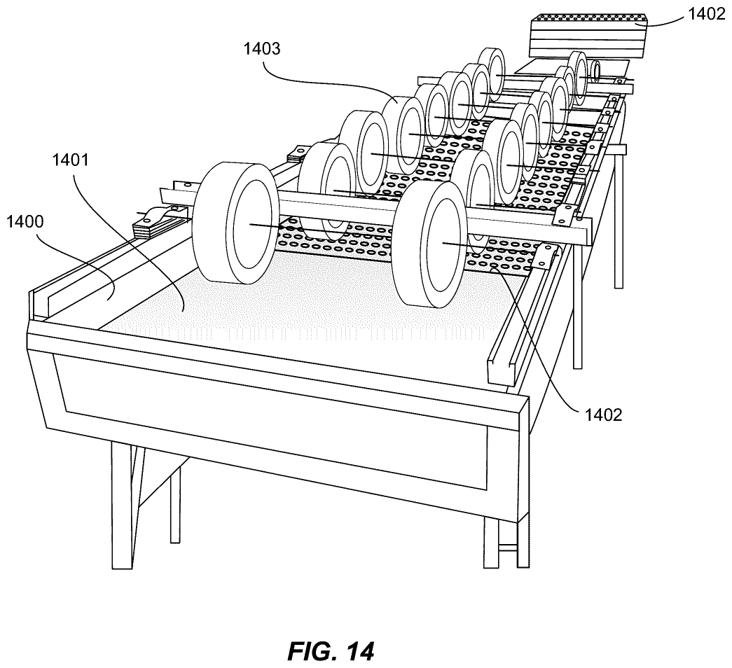

Similarly, after separating the trays from the tub after harvesting (Step F), any remaining stems and roots are cut from the growing substrate 1250 to leave the dirty trays 1245 which are washed and sanitized. In some embodiments, the washing step may include washing the trays with water (and/or desired solution) and cleaning the trays with a chemical cleaner. Step (G) In one embodiment, there is described a washing tray table filled with a chemical cleaner. The trays may be submerged in the chemical cleaner. FIG. 14, discussed in more detail below, is an example of an apparatus or part of an apparatus that can be used to wash trays.

Trays can be first washed with water (or other solution) prior to chemical cleaning (i.e. washing trays with chemical cleaners). After trays have been used at least once, dirt, debris, film, or other build up may accumulate on the tray. “On the tray,” in this context, means on any part of the tray, including but not limited to the top, bottom, sides, and on the inside and outside of the tray cells. Examples of build-up may include excess plant matter, growth media, algae growth, slime, residue, or other films or matter that may accumulate on the tray while used in the hydroponic system. The purpose of this washing is to remove any dirt, debris, film, or other build up on the trays. It may be done with water alone or with water and soap solutions, for example. Removing build-up enables the subsequent steps to sterilize the trays more effectively. The trays may be power washer, rinsed, soaked, submerged, sprayed, and/or scrubbed. In one embodiment, the trays are power washed using a high-power sprayer.

In some embodiments, after water washing, the trays are then washed using chemical cleaners. In this step, trays are submerged into, coated with, filled with, covered with, rinsed with, and/or surrounded by chemical cleaners. Chemical cleaners may be in a liquid, semiliquid, vaporized, or gas state. Chemical cleans can include, for example, soaps, alcohols, detergents, acids, or bases depending on the type of tray and matter to be removed from tray. Further examples may include hydrogen peroxides, bleaches (sodium hypochlorite), quaternary ammonium solutions, low foaming alkaline detergents, peracetic acid, or combinations thereof. The chemical cleaner may be diluted as necessary to ensure safety for people, plants, and trays. For example, chemical cleaners should not be so corrosive that they melt or damage the tray, making the tray unusable in a hydroponic system.

In some embodiments, low foaming alkaline detergent, such as Master MHW, is beneficial because it is formulated to emulsify dirt, oils, and organic materials such as biofilm, without producing excessive foam from the high agitation of automatic washers. A quaternary ammonium compound, such as Kleengrow, may also be used to wash trays either alone or alongside Master MHW, because it offers long lasting and effective broad-spectrum microbial control. Quaternary ammonium compounds' mode of action is membrane disruption by denaturing proteins, which makes the compounds ideal for removing biofilms for long-lasting microbial control. The positively charged chemistry attacks negatively charged pests found on trays. Contrary to other chemicals, the performance is not impaired by pH changes or exposure to light or temperatures used to maintain plant growth. Because quaternary ammonium compounds are very stable the use of Kleengrow leaves approximately a 30-day residual on all propagation trays. The use of Sanidate 5.0, a hydrogen peroxide and peracetic acid-based sanitizer, can also be used to wash trays and remove biofilm; however, the solution is much less stable compared to the use of a quaternary ammonium compound sanitizer, and the cleaning solutions degrades quickly.

In some embodiments, the growth trays are first rinsed with a chemical cleaner and then submerged into a bath of a chemical cleaner. In one example, rinsed with a quaternary ammonium sanitizer spray, and then submerged in a high concentration quaternary ammonium solution for under 1 minute. The trays may be submerged into a high concentration quaternary ammonium solution, or other chemical cleaner, less than 10 minutes. The amount of time will depend on the type of cleaner being used, the concentration of the cleaner, and the make-up of the tray. If the cleaner is particularly tray-friendly (meaning it will not damage the tray), then the tray may be left in the cleaner for longer periods of time (overnight for example).

In another embodiment, trays are cleaned with Master MHW, a low foaming alkaline detergent, and Kleengrow, a quaternary ammonium compound solution. Master MHW may be diluted at approximately 1 oz to approximately 3 oz per gallon of water. Kleengrow may be diluted at approximately 0.25 to approximately 0.50 oz per gallon of water, which correlates to approximately 150 to approximately 300 ppm quaternary ammonium compound. Preferably, tray washing uses solutions at approximately 200 ppm quaternary ammonium compound. A concentration of any higher than approximately 300 ppm would not be considered appropriate for treating the trays when the sanitizing solution is not rinsed off before planting. Anything higher than 300 ppm could leave behind excess residue that would negatively impact propagation. A concentration of less than approximately 150 would not be effective in breaking down bacterial biofilms.

The trays may be optionally rinsed off with water after chemical cleaning to ensure no harmful chemicals impact the growth systems and plants. Water may be filtered, sterilized, deuterated, distilled, and/or tap. As described in more detail below, for the subsequent dielectric step to efficiently kill pathogens, the inventors have found that the trays must not be too wet or too dry. Therefore, after the washing step, the trays are dried to a desired moisture content.

In some embodiments, the washed trays are thoroughly dried to near zero moisture 1255, prior to being reused for the next growth cycle. In some embodiments, the total moisture content may be less than about 5%, such as less than 4%, 3%, 2% or even less than 1%. Percent moisture may be determined using moisture content readers, spectrology, or through comparing the tray weight before and after washing. If weight is used to determine percent moisture, the trays may be measured individually or in groups. The trays may also be randomly sampled and tested.

The desired moisture content may also be determined and evaluated through physical inspection of the trays, such as whether the tray is dry to the touch or has no beads or pools of water. A tray that is dry to the touch or that has no beads or pools of water will have a percent moisture that is high enough to enable pathogen killing in the dielectric step but low enough to prevent tray damage.

The features and advantages of the tubs, methods of growing and systems used for growing disclosed herein are illustrated by the following example, which is not to be construed as limiting the scope of the present disclosure in any way.

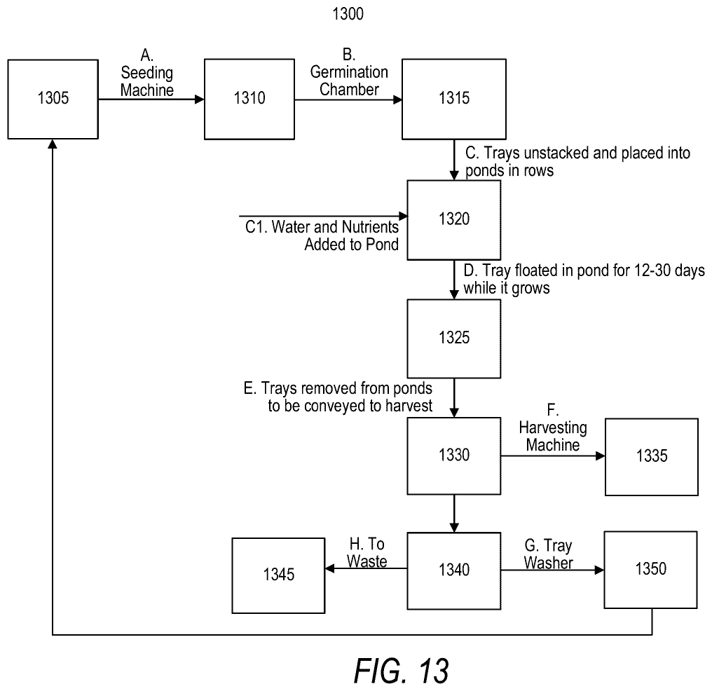

In contrast to the mobile tub system illustrated in FIG. 12, that is consistent with some inventive embodiments, FIG. 13 is a flow diagram of a pond system for hydroponic plant growth 1300. The pond system is also based on a process that starts with a cleaned and sanitized growth trays 1305 that are used in seeding step (A). In some embodiments, a seeding machine may be used to automate the process of planting seeds or seedlings in step (A).

As described herein, seeds are typically sown in trays, flats, or seedling beds filled with a growing medium, such as nutrient-rich soil. In some embodiments, growth trays for hydroponics can be made of durable and food-safe materials, such as plastic or Styrofoam. These materials are lightweight, easy to clean, and resistant to water and nutrient solutions. In some embodiments, the growth trays come in various sizes and shapes, and are typically rectangular or square.

After seeding, the seeds are germinated in Step (B) by placing the growth trays 1310 into a germination chamber, as previously described.

After the germination step (B) is complete, resulting in growth trays comprising sprouted seedlings, the trays 1315 are removed from the germination chamber and floated in ponds in which water and nutrients have been added 1320. The sprouted seedlings trays are floated in these ponds for 12-30 days, depending on the plant.

Once the plant has grown to maturity, it is ready for the harvesting 1325 and Step E. In some embodiments, harvesting is performed using a harvest machine. After harvesting 1230, the harvested plant is moved to cold storage 1235. After harvesting, any remaining stems and roots are cut from the growing substrate to leave the dirty trays 1245 which are washed, sanitized and dried to a desired moisture level 1250, prior to being reused for the next growth cycle.

As stated, FIG. 14 is an example of an apparatus or part of an apparatus that can be used to wash trays. The washing tray table (1400) may be filled with a chemical cleaner (1401). The trays (1402) may be submerged in the chemical cleaner and wheels (1403) may prevent the trays from floating on top of the chemical cleaner. The washing tray table may be operated by hand or may be part of an automated system.

### Drying Step

For the subsequent dielectric step to efficiently kill pathogens, the inventors have found that the trays must not be too wet or too dry. Therefore, after the washing step, the trays are dried to a desire moisture content. The dielectric apparatus can damage trays that are too wet or too dry, causing them to break and become unusable. A moisture content that is too low can allow pathogens to “dry up” as well, enabling them become dormant, escape the killing effects of the dielectric apparatus, and then revive themselves once rehydrated when reintroduced into the hydroponic system. A moisture content that is too high can cause the trays to crack, break, or become damaged during the dielectric step.

A desired total moisture content may be about 5%, about 10%, about 15%, about 20%, about 25%, or about 30%. The moisture content does may also include a range between any of the proceeding values, including the value as an approximately end point. For those values in this application, “about” or “approximately” means±3%. Percent moisture may be determined using moisture content readers, spectrology, or through comparing the tray weight before and after washing. If weight is used to determine percent moisture, the trays may be measured individually or in groups. The trays may also be randomly sampled and tested.

The desired moisture content may also be determined and evaluated through physical inspection of the trays, such as whether the tray is dry to the touch or has no beads or pools of water. A tray that is dry to the touch or that has no beads or pools of water will have a percent moisture that is high enough to enable pathogen killing in the dielectric step but low enough to prevent tray damage.

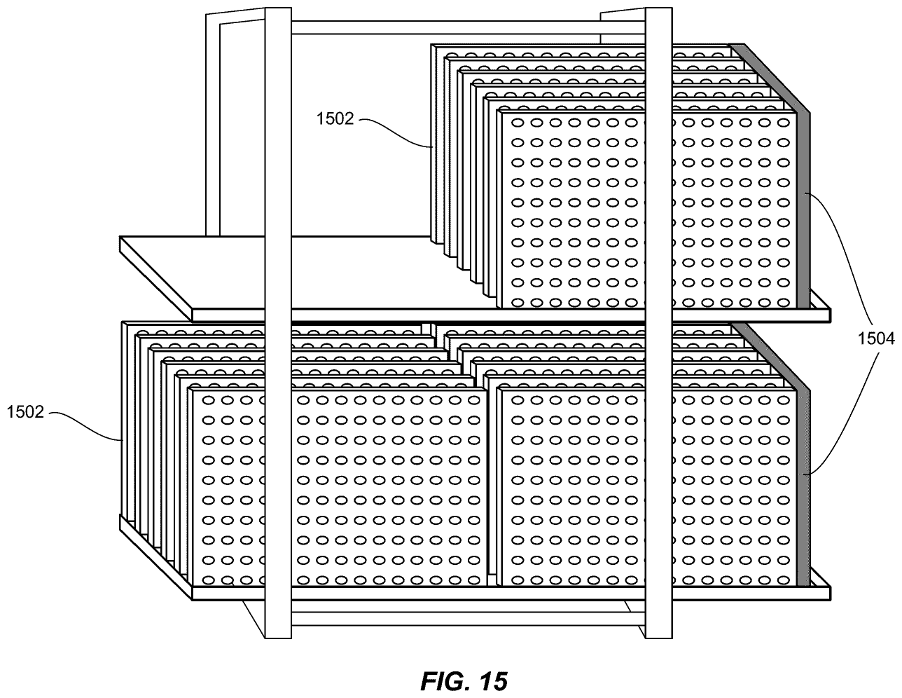

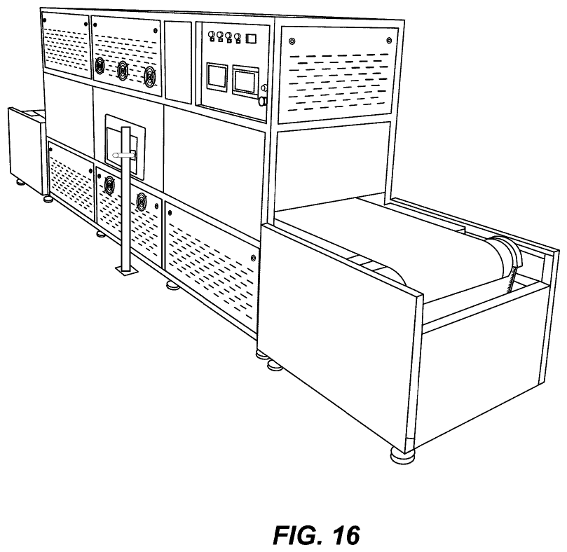

After trays are washed, they may be moved to a drying rack. The drying rack may contain fans, or other air stream devices, that push air through the trays to support the evaporation of water. Trays may be shaken dry or left on a drying rack without any additional drying aids. One embodiment of a tray drying apparatus is depicted in FIG. 15, where trays rest on a drying rack and are dried using box fans (1504). The trays should be placed with enough space in between then to allow for air flow. The position of the box fans (1504) is adjustable. For example, the box fans, or any other air stream devices, may be place to the left, right, below or on top of the trays.

Once the trays are at a desired moisture content, the trays can be moved into a dielectric apparatus.

### Dielectric Step

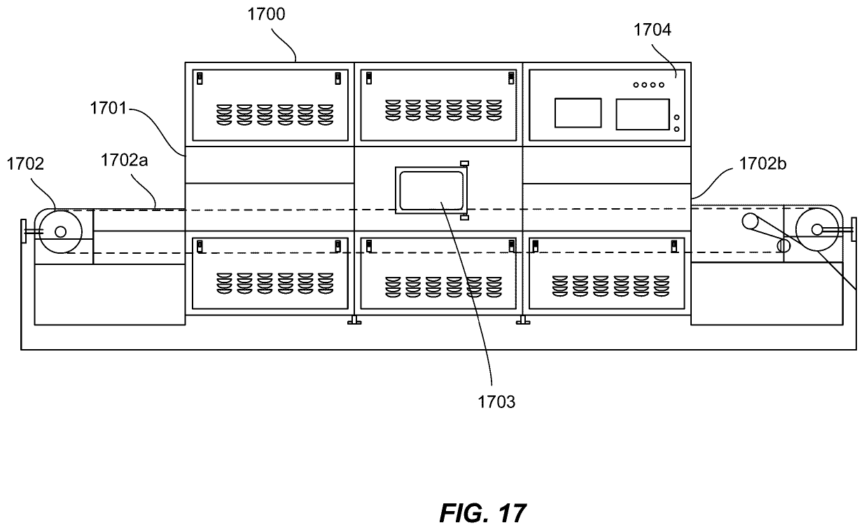

The purpose of the dielectric step is to kill pathogens that escaped the washing step. Dielectric devices are capable of emitting electromagnetic radiation in the form of, for example, radio-frequency or microwaves. S. Nelson et al., Radio-Frequency Heating of Alfalfa Seed for Reducing Human Pathogens, 45 American Society of Agricultural Engineers 1937 (2002); S. Nelson et al., A Half Century of Research on Agricultural Applications for RF and Microwave Dielectric Heating, 2011 ASABE Annual International Meeting. A common household microwave functions by applying microwaves to food causing water molecules within the food to vibrate and produce heat. In certain circumstances, when enough power is applied to the food, it may boil or burst. Inventors found that this concept may be applied to pathogens in growth trays. While not necessarily wishing to be bound to this theory, when pathogens are subjected to sufficient dielectric radiation they burst much like overheated food. This kills the pathogens. Inventors discovered that running trays with a desired moisture content through a dielectric apparatus, such as shown in FIGS. 16 and 17, drastically reduces pathogens in trays. Implementing these methods has improved crop health and yield because they reduce pathogens being introduced into growth ponds.

In one embodiment, a dielectric apparatus is capable of emitting electromagnetic radiation. It has a control panel allowing a user to control and/or change the settings of the apparatus, such as speed of a conveyor when the growth trays are placed on a conveyor belt. The apparatus may have multiple emitters within it that are independently adjustable to change the amount of power used via control panel. The number of emitters allow it to operate at different power levels. For example, when the apparatus has four emitters, it is operating at full power. Three emitters indicated the apparatus was operating at 75% full power; two emitters meant 50% full power; and one emitter meant 25% full power. The apparatus may have a power of from about 0.6 kW to about 12 kW. In one embodiment, the apparatus has a power of about 10 kW and comprises at least one emitter, at least two emitters, at least three emitters, or at least four emitters. The apparatus may have an output frequency of up to approximately 2500 MHz. In one embodiment, the apparatus has an output frequency of up to approximately 2450 MHz±50 MHz.

In an embodiment, as depicted in FIGS. 16 and 17, the dielectric apparatus that may optionally have a conveyer belt (1702) within the body of apparatus that advances trays from one end of the belt (1702a) to another (1702b). The body of the apparatus (1700) may be solid or may have one or more doors or windows (1703) on it that allow trays to be monitored, adjusted, or removed from the apparatus before they reach the end of the belt. It may also have a control panel (1704) to adjust the settings of the apparatus. The belt may have a transmission speed of about 0-20 m/min and may be adjustable. In one embodiment, the transmission speed may be 0-5 m/min. The apparatus may have multiple emitters within it that are independently adjustable to change the amount of power used. In one embodiment, the apparatus has a power of about 10 kW. The apparatus may have an output frequency of up to approximately 2500 MHz. In one embodiment, the apparatus has an output frequency of up to approximately 2450 MHz±50 MHz. Any number of emitters may be used to achieve the desired power levels as described above.

As further described in the experiments below, microwaving trays efficiently kills pathogens in trays that survived chemical cleaning. Such methods enable a broad range pathogen killing. Pathogens may include, but are not limited to, water molds, oomycetes, fungus, or bacteria. Pythium spp. and Phytophthora spp. are common oomycetes present in hydroponic systems that attack plant roots.

### Ultrasound

At any point throughout this process, the trays may optionally be subjected to ultrasonic currents. In one embodiment, ultrasound is applied to the trays during the washing step while trays are submerged in a bath of chemical cleaner.

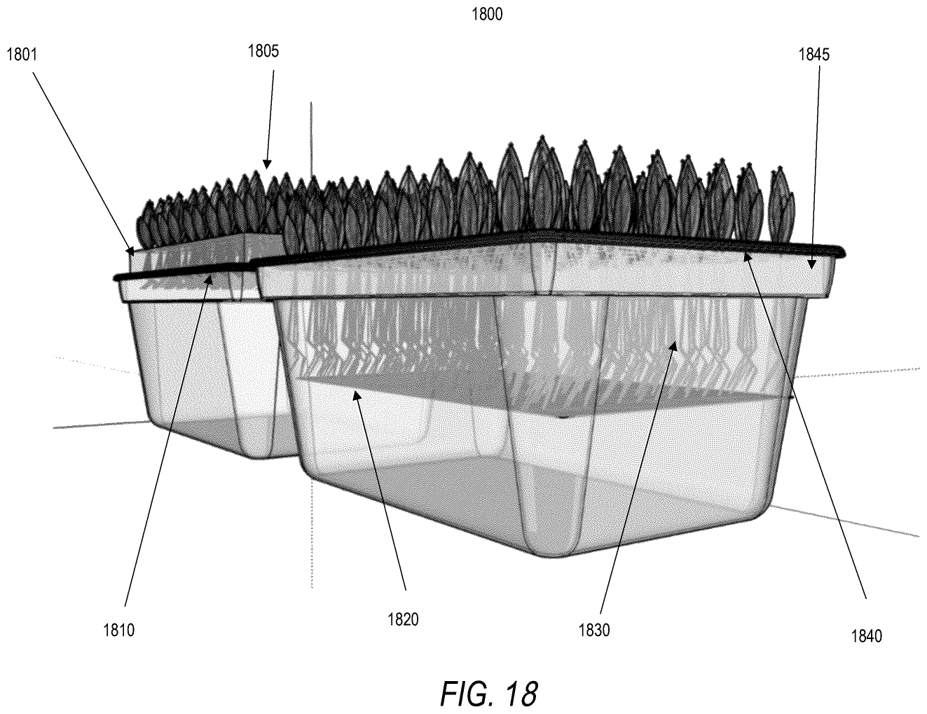

FIG. 18 shows the case in which the top raft 1801 floats on nutrient rich water as plants develop. Typically, for the first several days when plants 1805 and roots 1810 are young, they need to be immersed in nutrient rich water. As the water level 1820 drops due to usage by the plants and evaporation, the floating raft 1840 settles down into a specially designed slot in the tub 1845. From that point on, the water level 1820 will drop below the bottom of the floating raft. The roots 1830 will follow the water level 1820 down and the highest parts of the roots 1830 will be exposed to air. The raft 1840 will settle into the slots at a level that will allow the plants to be commercially harvested through a machine (not shown) that trims off the upper parts of the plants.

In one embodiment, the tub has a connection hose that allows it to be connected to a source of air that feeds a bubbler that is fitted into the bottom of the tub. The bubbler is sized to fit the specially designed tub so that the bubbles emitted generate turbulence throughout the root system of the leafy greens. This turbulence is an important part of the disclosed process. Without being bound by theory, the turbulence caused by the bubbler is expected to prevent bacteria and disease from attaching to the root system. The turbulence caused by the bubbler is also expected to help in removing extrudates that are emitted from the roots and are the food source of bacteria and disease. For at least these reasons, the bubblers run for most of the growth process, and in some embodiments, constantly throughout the growth process.

In other embodiments, there is disclosed other growing methods that speed up the growth cycle of the plant to reduce the time that disease has to develop. For example, in an embodiment, supplemental lighting is used to speed up the growth process. Similarly, nutrient levels in the water sources are carefully balanced to optimize growth of the plant. In addition, temperatures of the water and air above the plants are controlled to achieve optimum growth characteristics.

After the growth period (which in some cases is as low as 14 days), the plants are harvested and the tubs and rafts are completely sanitized. Plants being grown in isolated batches, with constant bubbling, and complete sanitations after every growth cycle is described herein. In one embodiment, the disclosed tubs are arranged in special racks that allow two or more tubs to be connected. In one example, there could be 6 to 12 tubs in one rack. Reference is made to FIG. 6. Here it shows that the tubs described herein 610 are located in racks 675, that are on wheels 680 so they can be moved into an area where they are butted up against each other to form a large field of solid growing plants. The system described herein has multiple rollers 685 that allow racks of tubs to be on wheels that roll on concrete or wheels that roll on rails.

The system described herein enables the movement of the racks throughout the life cycle of plant growth. In one embodiment, a plurality of tubs, such as in groups of 5-15, such as 6-12 or 5-12 sit on a platform that has a common air-line and drain. This platform is referred to as a “row”. Rows can be conveyed throughout the greenhouse on rails with pneumatic lifts. As shown in FIGS. 6 and 7, a mechanized system to move the rows or racks of tubs through the growing system, into the harvest system, into the cleaning and sanitizing system, into the seeding system, and back into the growing area is described. This system allows a large greenhouse, such as a ten (10) acre greenhouse, to operate efficiently without a large labor pool.

In some embodiments, the disclosed invention comprising a moving tub system with distributed air is best appreciated by contrasting it to currently available commercial growing systems. For crops like leafy greens and herbs, it is ideal to have a centralized area for seedling/germination and for harvest/packaging and other inter-crop cycle activities—this is commonly referred to as the “headhouse”. That means that the plants themselves must move from the “headhouse” to the greenhouse where they'll grow to maturity before returning to the “headhouse” to be processed.

Once plants go into the greenhouse to grow, they need light, air, and water. The greenhouse structure provides for the air and light plants need. But there are several different strategies growers use to deliver water to their plants. These include (1) overhead irrigation by moving booms and/or fixed misters; (2) drip line/tube and/or emitters where water is delivered to plants through distributed lines at low flow rates through pressure compensated emitters. This is typically used for plants that will remain stationary for a long time, such as cucumbers, strawberries, and tomatoes as well as cannabis. This method requires significant labor in setting up drip tube/emitters and they frequently need to be flushed to avoid buildup/clogging; (3) Flood and drain systems, which is commonly used for longer cycle ornamental plants when implemented on the greenhouse floor; (4) NFT troughs, in which water is trickled through a gutter—intermittently or continuously through low flow tubes that run throughout the greenhouse; and (5) Deep water culture (DWC) ponds, in which large ponds filled with recirculating nutrient solution both convey the plants in floating trays and provide them nutrient water as they grow. These systems are difficult to clean and since water-borne root pathogens can move freely throughout the pond, they are non-ideal for sensitive root crops. In addition, ponds are costly to shut down if a breakout of infection occurs and complete cleaning is required.

The Fully-Quarantined Mobile Tub System described herein is different from the aforementioned systems in the fundamental way that it does not rely on the movement of water to irrigate the plants throughout the crop cycle. Instead of pumping fertilized nutrient solution throughout the greenhouse, there is described herein a system for conveying only low-pressure air throughout the greenhouse to deliver turbulence to the nutrient solution in the root zone. In some embodiments, the airlines in the disclosed system contain a constantly fast flowing stream of warm, dry air and because of this they are not a suitable environment for pathogen growth to flourish. This stands in stark contrast to the plumbing systems of other systems that are ambient temperature, wet, and contain all the nutrients necessary for the growth of algae and other microbes within the irrigation lines and adjacent to them.

In the inventive system, once the tubs are filled at the beginning of the crop cycle, no water will enter the tub and none will leave except by way of evaporation and biomass accumulation. After filling, the tub, attached to a rolling table with other self-contained tubs, is then sent into the greenhouse with all the water it needs to reach maturity. This fundamental difference makes it a much more robust method for mitigating spread of water-borne pathogens. It has also been discovered that the vigorous agitation accomplished with the large air-diffuser and high air-flow rate provide additional protection against root infection and promotes vigorous growth. The disclosed design and configuration thus achieves important benefits for growing leafy greens and herbs, especially for mechanical harvest.

The system described herein comprises tables that are significantly heavier than traditional horticulture techniques used to grow plotted plants. Traditional, “Dutch rolling tables” are versatile table and rail systems that are popular for growing potted plants. Unlike the described Fully-Quarantined Mobile Tub System, Dutch rolling tables used a watering system with drip or overhead booms as well as herbs in ebb and flow setups.

The system described herein that comprises tables that are significantly heavier than traditional horticulture techniques can efficiently move larger loads, such as the described tubs, on large tables from the processing areas down conveyors and into greenhouse bays using pneumatic cylinders to change to the perpendicular direction. In some embodiments, the described tables can be configured to feed the grown plants through a harvesting machine.

In some embodiments, tables used in the present disclosure may be fabricated from aluminum extrusions. In some embodiments, the frame has two aluminum pieces, such as 4×2″ pieces, that run the length of the table which are connected by cross members on both the top and bottom of the tray. This edge allows the table to roll on the conveyor the runs perpendicular to the greenhouse bays.

In some embodiments, on the underside of the table there may be 3 cross members that are both structural and are also the locations of the wheel mounts. In some embodiments, there may be two types of wheels on the bottom of the tables: guide wheels and drive wheels. The guide wheels grip the rail from the side to keep the table moving straight, and the drive wheels just roll on top of the rail. The middle cross member has two drive wheels while the cross members on each side have two guide wheels. Thus, in some embodiments, each table may have 6 wheels (2 guide, 4 drive) on its cross members. These wheels allow the table to roll on the rails within a greenhouse bay.

The top of the table may contain two (2) lighter gauge cross members (rungs) to support each tub. In some embodiments, these rungs have holes drilled to match those of the tubs so that the tub can be bolted to the table rungs with a watertight connection.

In some embodiments, the table contains an integrated air manifold pipe. For example, in some embodiments, there is an air manifold located between the wheel cross members and rungs. In some embodiments, this manifold may be constructed of ¾″ Schedule 80 PVC pipe, and this pipe runs the length of the table. For each tub, the ¾″ pipe is tapped and a hose barb is inserted. A small diameter (¼″ ID) hose connects the barb at each tub to the diffuser in the bottom of the tub.

In some embodiments, at one end of the table manifold, there is a removable cap that allows the manifold to be cleaned out/cleared. At the other end of the manifold, there is a connection to attach the manifold to the air mainline.

In some embodiments, the table contains an integrated air manifold pipe. In some the integrated air manifold pipe may be attached to an air main. For example, the air main line may be a 2″ Schedule 80 PVC pipe fed from a single blower. A 3.5 HP blower could be used to supply air to up to 25 tables at once so that a single 200 ft greenhouse bay may have 3 or more blowers to feed 60-70 tables (600-700 tubs). For each table in the bay, the air main is tapped and a longer ¾″ hose attaches the table to the air main via a threaded connection. The table with the air manifold was configured to deliver air to each tub's diffuser. In some embodiments, the manifold is located under each table with a connection to the air main to connect an entire table to the airline with one connection.

In some embodiments, the above-described table is used in conjunction with the tray and tub described herein, which allowed the tub that contained a large diffuser and configured a lip portion to hold tray in place.

In some embodiments, there is described a system in which the disclosed table is configured to be fed through a harvester without handling trays. More generally, the system includes one or more tables configured in a row, that are constructed from aluminum onto which the tubs are bolted. The air manifold is located underneath the table that feeds each tub's diffuser, which is screwed into the bottom of each tub. As stated, the air manifold can be connected to the air “main” that feeds each row of tables i.e. one (dis)connection each time a table is relocated. In the disclosed system, the tables have 2-4 sets of wheels underneath them so that they can roll on rails down the bay of a greenhouse. In some embodiments, at the ends of the bays, conveyor wheels grip the tables on their undersides to move them in the perpendicular direction. As a result of the design of the tub and the ending position of the trays within the tubs, at harvest the entire row is conveyed through the harvesting machine with the trays.

The features and advantages of the present invention are more fully shown by the following examples which are provided for purposes of illustration and are not to be construed as limiting the invention in any way.

### Example

### Example 1—Bacteria

Inventors first conducted initial tests of the Styrofoam trays to get an idea of the micro count of bacteria, and the amount of time trays needed to be expose to electromagnetic radiation for a good log reduction of bacteria. To conduct the test trays were submerged in dirty rice water to pick up a micro load of bacteria. It is known that multiple types of bacteria are present in dirty or washed rice water. The same dirty rice water was used in each test. Micro loads were measured using Aerobic Plate Count (APC). APC tests are not specific to a type of bacteria and enable the growth of many types of bacteria. This made APC tests a measure for measuring a comprehensive kill of all the types of bacteria present in dirty rice water. The trays were then dried or not dried before being heated in a device that had approximately 1050 W of power.

Two types of tests were conducted: wet tests and dry tests. In wet tests, the trays were submerged and then immediately tested. In dry tests, the trays were submerged, dried overnight, and or were shaken to remove excess water. Later experiments used high pressure air to remove water from the tray. In each experiment, trays were swabbed at time zero before any microwaving, drying, or shaking.

In Wet Test 1, trays were 1 heated for 2 minutes, swabbed, and then heated for an additional 2 minutes (for a total of 4). In Wet Test 2, trays were heated for 4 consecutive minutes. In Wet Test 3, shortened the heating times that were tested. Trays were swabbed before heating, then swabbed at 1 minute, at 1.5 minutes, at 2 minutes, and after 2.5 minutes. Wet Tests 4, 5, and 6 repeated the experiment of Wet Test 3. Results of the tests can be found in Tables below. Power was kept constant throughout the experiments.

Wet Tests

| Test No. | Heating Time (min.) | APC |
|---|---|---|
| 1 | 0 | 5,390,00 |
| | 2 | 2,800 |
| | 4 | 190 |
| 2 | 0 | 4,070,000 |
| | 4 | 370 |
| 3 | 0 | 204,000 |
| | 1 | 170 |
| | 1.5 | 140 |
| | 2 | 130 |
| | 2.5 | 100 |
| 4 | 0 | 200,000 |
| | 1 | 370 |
| | 1.5 | 190 |
| | 2 | 180 |
| | 2.5 | 80 |
| 5 | 0 | 2,860,000 |
| | 1 | 60 |
| | 1.5 | 10 |
| | 2 | <10 |
| | 2.5 | <10 |
| 6 | 0 | 1,9000,000 |
| | 1 | 7,500 |
| | 1.5 | 10 |
| | 2 | 30 |
| | 2.5 | <10 |

West Test Averages

| Heating Time (min.) | APC |
|---|---|
| 0 | 1,291,000 |
| 1 | 2,025 |
| 1.5 | 88 |
| 2 | 8 |
| 2.5 | 50 |

In Dry Test 1, after the trays were submerged in rice water, the trays were dried for 24 hours before being heated. Trays were swabbed while wet, after 24 hours of drying, after 2 minutes of being heated, then again after an additional 2 minutes for 4 total minutes. Dry Test 2 repeated Dry Test 1. In Dry Test 3, the trays were shaken to remove excess water from them instead of being dried for 24 hours. The testing and swabbing were then conducted in the same manner as the shortened heating times as in the wet tests. Dry Test 4 was a repeat of Dry Test 3. Results of the tests can be found in Tables below.

Dry Test

| Test No. | Dry Time (hr.) | HeatingTime (Min.) | APC |
|---|---|---|---|
| 1 | 0 | 0 | 4,270,000 |
| | 24 | 0 | 1,630,000 |
| | 24 | 2 | 3,600 |
| | 24 | 4 | 1,350 |
| 2 | 0 | 0 | 720,000 |
| | 24 | 0 | 300,000 |
| | 24 | 2 | 3,000 |
| | 24 | 4 | 780 |
| 3 | 0 + Shake Off | 0 | 100,000 |
| | 0 + Shake Off | 1 | 450 |
| | 0 + Shake Off | 1.5 | 350 |
| | 0 + Shake Off | 2 | 160 |
| | 0 + Shake Off | 2.5 | 20 |
| 4 | 0 + Shake Off | 0 | 400,000 |
| | 0 + Shake Off | 1 | 900 |
| | 0 + Shake Off | 1.5 | 160 |
| | 0 + Shake Off | 2 | 10 |
| | 0 + Shake Off | 2.5 | <10 |

Shaken Dry Test Averages

| Heating Time (min.) | |
|---|---|
| (after shake off) | APC |
| 0 | 250,000 |
| 1 | 270 |
| 1.5 | 255 |
| 2 | 85 |
| 2.5 | 15 |

24 Hr. Dry Test Averages

| Dry Time (hr.) | Heating Time (min.) | APC |
|---|---|---|
| 0 | 0 | 2,495,000 |
| 24 | 0 | 965,000 |
| 24 | 2 | 3,300 |
| 24 | 4 | 1,065 |

### Experiments—Fungi

Tests were conducted to determine that trays were responsible for introducing pathogens into the hydroponic growth systems and that heating treatment could kill pathogens. Trays, plants, soil, water, and pond were sampled, and samples were cultured and tested using Bait/PAR methods for species of Pythium and Phytophthora. Table: Plug Trays New v. Old reveals that no Pythium and Phytophthora are present in new trays, but Pythium was found in trays after use.

Plug Trays New v. Old

| | | Pythium Spp. | Phytophthora |
|---|---|---|---|
| No. | Sample ID | Present? | Spp. Present? |
| 1 | EPS Tray 1 [C] | No | No |
| 2 | EPS Tray 2 [B1] | Yes | No |

Table: Soil Baiting demonstrated that Pythium and Phytophthora are not present in the soil throughout the growing processing. This indicates that these pathogens are not introduced into the trays through the soil.

Soil Baiting: Spinach

| | | Pythium Spp. | Phytophthora |
|---|---|---|---|
| No. | Sample ID | Present? | Spp. Present? |
| 1 | Soil Sample 1 | No | No |
| | (Tower) | | |
| 2 | Soil Sample 1 | No | No |
| | (Hopper) | | |
| 3 | Soil Sample 1 | No | No |
| | (Wave) | | |

Pythium spp. were consistently isolated from samples #1 and #2 on semi-selective oomycete isolation media (PAR: see column #3 in results' table).

Water Samples: Spinach

| | | Pythium Spp. | Phytophthora |
|---|---|---|---|
| No. | Sample ID | Present? | Spp. Present? |
| 1 | Water Sample 15 | Yes | No |
| 2 | Water Sample 3 | Yes | No |

Trays were sampled after being used at least once. Trays tested positive for species of Pythium. The trays were then run through a dielectric device three times and sampled after each run. No Pythium was detected in the trays after the first run (and subsequent runs), demonstrating microwaving trays can kill pathogens superior to standard sterilization methods alone.

EPS Plug Trays: Spinach

| | | Pythium Spp. |
|---|---|---|
| No. | Sample ID | Present? |
| 1 | Control | Yes |
| 2 | Wave 1 | No |
| 3 | Wave 2 | No |
| 4 | Wave 3 | No |

### Yield Experiments

In these experiments, both heated and non-heated trays were power washed, rinsed with a quaternary ammonium sanitizer spray, and then submerged in a high concentration quaternary ammonium solution for under 1 minute. The trays to be heated were air-dried on a rack until they were dry to the touch (approximately 5% moisture) before they pass through the dielectric device. The trays were seeded the day of cleaning/sanitizing or the day after. Once the seeds germinated (and some cotyledons were visible), the trays were placed on to a first production pond. After approximately 7 days, they were transferred to a second production pond; the exact date of transfer depended on the type of crop. Plants were harvested approximately 12-22 days after plants were first introduced into the ponds depending on type of crop and appearance of growth. Tests were completed using spinach, arugula, and lettuce. However, these methods may be applied to a wide variety of crops capable of being grown hydroponically.

Different heating parameters were tested in these experiments. The dielectric apparatus used in this experiment had 4 emitters and a production belt. The number of emitters signified the amount of radiation power the trays were subjected to during treatment. Heating treatments conducted with 4 emitters indicate that the apparatus was operating at full power. Three emitters indicated the apparatus was operating at 75% full power; two emitters meant 50% full power; and one emitter meant 25% full power. The frequency of the production belt is reported in Hertz (Hz). DUL means Days Under Light.

Spinach Experiments

| | Media | | Dielectric Oven | Total Net | Yield | |
|---|---|---|---|---|---|---|
| DUL | Type | Rows | Treatment | Weight | (g/tray) | Increased? |
| 17 | Lam. + | 4 | 4 @ 20 Hz | 17,600 | 367 | Yes |
| | Pro | | | | | |
| 17 | Lam. + | 6 | No | 22,400 | 311 | |
| | Pro | | | | | |
| 16 | BX + | 4 | 4 @ 28 Hz | 20,200 | 421 | Yes |
| | M + | | | | | |
| | PGX | | | | | |
| 16 | BX + | 2 | No - New | 9,800 | 408 | |
| | M + | | Trays, 1st | | | |
| | PGX | | Cycle | | | |
| 17 | BX + | 2 | 4 @ 28 Hz | 8,700 | 363 | Yes |
| | M + | | | | | |
| | PGX | | | | | |
| 17 | BX + | 4 | No - New | 12,750 | 266 | |
| | M + | | Trays, 1st | | | |
| | PGX | | Cycle | | | |
| 16 | BX + | 6.75 | 4 @ 28 Hz | 46,850 | 578 | Yes |
| | M + | | | | | |
| | PGX | | | | | |
| 16 | BX + | 7.25 | No - New | 42,800 | 492 | |
| | M + | | Trays, 1st | | | |
| | PGX | | Cycle | | | |
| 17 | BX + | 6 | 4 @ 28 Hz | 32,900 | 457 | Yes |
| | M + | | | | | |
| | PGX | | | | | |
| 17 | BX + | 5 | No | 20,450 | 341 | |
| | M + | | | | | |
| | PGX | | | | | |
| 17 | BX + | 4 | 4 @ 28 Hz | 14,650 | 305 | Yes |
| | M + | | | | | |
| | PGX | | | | | |
| 17 | BX + | 1 | No | 2,450 | 204 | |
| | M + | | | | | |
| | PGX | | | | | |
| 17 | BX + | 4 | 4 @ 28 Hz | 14,650 | 305 | Yes |
| | M + | | | | | |
| | PGX | | | | | |
| 17 | BX + | 4 | No- New | 9,850 | 205 | |
| | M + | | Trays, 1st | | | |
| | PGX | | Cycle | | | |
| 16 | BX + | 1 | 4 @ 28 Hz | 4,350 | 363 | Yes |
| | M + | | | | | |
| | PGX | | | | | |
| 16 | BX + | 6 | No | 24,150 | 335 | |
| | M + | | | | | |
| | PGX | | | | | |
| 16 | BX + | 4 | 4 @ 30 Hz | 18,250 | 380 | Yes |
| | M + | | | | | |
| | PGX | | | | | |
| 16 | BX + | 4 | No | 15,250 | 318 | |
| | M + | | | | | |
| | PGX | | | | | |
| 16 | BX + | 4 | 4 @ 30 Hz | 18,250 | 380 | Yes |
| | M + | | | | | |
| | PGX | | | | | |
| 16 | BX + | 2 | No | 6,900 | 288 | |
| | M + | | | | | |
| | PGX | | | | | |
| 16 | BX + | 2 | 4 @ 30 Hz | 6,250 | 260 | No |
| | M + | | | | | |
| | PGX | | | | | |
| 16 | BX + | 4 | No | 15,250 | 318 | |
| | M + | | | | | |
| | PGX | | | | | |
| 16 | BX + | 2 | 4 @ 30 Hz | 6,250 | 260 | No |
| | M + | | | | | |
| | PGX | | | | | |
| 16 | BX + | 2 | No | 6,900 | 288 | |
| | M + | | | | | |
| | PGX | | | | | |
| 12 | LAM − | 3 | 4 @ 30 Hz | 14,950 | 415 | Yes |
| | GPS | | | | | |
| 12 | LAM − | 3 | No | 13,750 | 382 | |
| | GPS | | | | | |
| 14 | BX + | 7 | 4 @ 30 Hz | 30,150 | 359 | Yes |
| | M + | | | | | |
| | PGX | | | | | |
| 14 | BX + | 7 | No | 28,900 | 344 | |
| | M + | | | | | |
| | PGX | | | | | |

Arugula Experiments

| | Media | | Dielectric Oven | Total Net | Yield | |
|---|---|---|---|---|---|---|
| DUL | Type | Rows | Treatment | Weight | (g/tray) | Increased? |
| 17 | ½ | 4 | 4 @30 Hz | 21,250 | 443 | Yes |
| | Lam. | | | | | |
| | ½ | | | | | |
| | Pro − | | | | | |
| | Mix | | | | | |
| 17 | ½ | 2 | | 5,750 | 240 | |
| | Lam. | | | | | |
| | ½ | | | | | |
| | Pro − | | | | | |
| | Mix | | | | | |
| 18 | Bx + m | 6 | 4 @ 28 Hz | 38,300 | 532 | No |
| | and | | | | | |
| | PGX | | | | | |
| 18 | Bx + m | 6 | | 43,200 | 600 | |
| | and | | | | | |
| | PGX | | | | | |
| 19 | Bx + m | 2 | 4 @ 28 Hz | 9,700 | 404 | Yes |
| | and | | | | | |
| | PGX | | | | | |
| 19 | Bx + m | 2 | | 6,750 | 281 | |
| | and | | | | | |
| | PGX | | | | | |
| 18 | Bx + m | 2 | 4 @ 28 Hz | 10,050 | 419 | No |
| | and | | | | | |
| | PGX | | | | | |
| 18 | Bx + m | 2 | | 10,750 | 448 | |
| | and | | | | | |
| | PGX | | | | | |
| 14 | Lam − | 2 | 4 @ 28 Hz | 8,500 | 354 | No |
| | GPS | | | | | |
| | & | | | | | |
| | Pro − | | | | | |
| | Mix | | | | | |
| 14 | Lam − | 3 | | 14,200 | 394 | |
| | GPS | | | | | |
| | & | | | | | |
| | Pro − | | | | | |
| | Mix | | | | | |
| 16 | Lam − | 2 | 4 @ 28 Hz | 14,000 | 583 | Yes |
| | GPS | | | | | |
| | & | | | | | |
| | Pro − | | | | | |
| | Mix | | | | | |
| 16 | Lam − | 4 | | 25,900 | 540 | |
| | GPS | | | | | |
| | & | | | | | |
| | Pro − | | | | | |
| | Mix | | | | | |
| 16 | Bx + m | 1 | 4 @ 28 Hz | 3,650 | 304 | Yes |
| | and | | | | | |
| | PGX | | | | | |
| 16 | Bx + m | 4 | | 14,050 | 293 | |
| | and | | | | | |
| | PGX | | | | | |
| 17 | Bx + m | 2 | 4 @ 28 Hz, | 10,100 | 421 | Yes |
| | and | | 2nd Cycle | | | |
| | PGX | | | | | |
| 17 | Bx + m | 2 | | 7,950 | 331 | |
| | and | | | | | |
| | PGX | | | | | |

Lettuce Experiments

| | Media | | Dielectric Oven | Total Net | Yield | |
|---|---|---|---|---|---|---|
| DUL | Type | Rows | Treatment | Weight | (g/tray) | Increased? |
| 22 | Pro − | 7 | 4 @ 28 Hz | 98,500 | 1173 | Yes |
| | Mix | | | | | |
| 22 | Pro − | 9 | | 106,600 | 987 | |
| | Mix | | | | | |

Average Yield

| | | Dielectric | | Total | Average |
|---|---|---|---|---|---|
| | Comp. | Oven | Average | Yield | Yield |
| Product | Entries | Treatment | DUL | (g) | (g/tray) |
| Spinach | 14 | Yes | 16 | 254,000 | 372.36 |
| | | No | | 231,600 | 321.43 |
| Arugula | 8 | Yes | 17 | 115550 | 432.50 |
| | | No | | 128,550 | 391 |
| Lettuce | 1 | Yes | 22 | 98,500 | 1173 |
| | | No | | 106,600 | 987 |

Disclosed embodiments may include any one of the following bullet-pointed features alone or in combination with one or more other bullet-pointed features, whether implemented as a device, system, apparatus, and/or method.
a method of sterilizing growth trays to kill pathogens.
the method comprising: washing growth trays with water at least once; applying at least one chemical cleaner to the trays; drying the trays to a desired percent moisture; and heating the trays using a dielectric apparatus that kills at least one pathogen.
a washing step performed using a power or high-pressure washer.
an application step comprising applying at least one chemical cleaner is performed while also subjecting the growth trays to ultrasonic energy.
an application step comprising applying at least one chemical cleaner comprises: rinsing the trays with a quaternary ammonium sanitizer spray, and then submerging the trays in a concentration of up to 300 ppm quaternary ammonium solution for less than 1 minute.
a desired percent moisture for the sterilized trays being less than 30% by weight.
a drying step that uses at least one fan and is used to achieve a moisture content ranging from 5 to 15%.
using a dielectric apparatus, such as a microwave.
a dielectric apparatus comprises a conveyor belt, a drying tunnel, a controlling system; wherein the conveyor belt is located inside the drying tunnel and the controlling system is attached to the drying tunnel; wherein the drying tunnel has emitters.
a microwave having at least four emitters that have a power of approximately 10 kW and an output frequency of approximately 2450 MHz.
a growth tray configured to contain and germinate plants.
a growth tray that is configured to sit and float on an aqueous solution that is contained in a tub.
a growth tray that has a top surface and a bottom surface with a plurality of openings completely through the top surface and the bottom surface, wherein the plurality of openings are configured to hold soil and germinate seeds in the soil.
a plurality of opening comprising at least three regions.
a top region having a top opening sufficient for sprouting plants to grow through the top surface and sidewalls having a tapered shape to a more narrow transition region.
a transition region having sidewalls that further taper to a more narrow end region.
an end region having straight sidewalls with a bottom opening for plant roots to grow through the bottom surface and contact the aqueous solution that is contained in a tub.
a growth tray is made of expanded polystyrene (EPS) and configured to sit and float on an aqueous solution that is contained in a tub.
a growth tray having a top surface and a bottom surface with 400 to 450 oval-shaped openings completely through the top surface and ending at a circular shaped opening at the bottom surface,
oval-shaped openings configured to hold soil and germinate seeds in the soil.
openings may comprise at least three regions: a top region having a top, oval-shaped openings having as its longest axis a diameter of 18-20 mm and tapered sidewalls having a taper angle of 2 to 4 degrees relative to a vertical plane drawn through the center of the openings.
a top region having a concaved shape bottom leading into a transition region.
a transition region typically has tapered sidewalls with a taper angle of 26 to 28 degrees relative to a vertical plane drawn through the center of the openings that lead to a more narrowed end region.
an end region typically has straight sidewalls with a bottom opening having a round shape and a diameter of 8-10 mm that allow plant roots to extend down through the bottom surface and contact the aqueous solution that is contained in a tub.
a tub configured to hold an aqueous mixture of water and a nutrient solution for growing plants.
a tub comprising a top portion having a rectangular shape and including a lip around the internal circumference that is configured to receive a growth tray; a bottom closed portion having a rectangular shape smaller in size than the top portion.
a bottom closed portion of the tub comprising at least one attachment mechanism for removably attaching the tub to a frame.
an air diffuser configured to create a turbulent flow of the aqueous mixture located in the tub.
Sidewalls that taper from the top portion to the bottom closed portion, wherein the tapered sidewalls are configured to allow the roots hanging from the growth tray to grow toward the center of the tub.
a system for hydroponically growing plants, whereby the plants are grown in isolated batches, comprising a growth tray and a tub, as described herein.
a method of hydroponically growing plants, comprising planting a plurality of seeds in growth media, where the growth media are contained in plurality of trays that are configured to float on top of a tub and to provide access for the crops to the water mixture when floating.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

We claim:

1. A method of sterilizing growth trays to kill pathogens comprising:
washing said growth trays with water at least once;
applying at least one chemical cleaner to the trays;
drying the trays to a desired percent moisture; and
heating the trays using a dielectric apparatus that kills at least one pathogen.

2. The method of claim 1, wherein said washing is performed using a power or high-pressure washer.

3. The method of claim 1, wherein said applying at least one chemical cleaner is performed while also subjecting the growth trays to ultrasonic energy.

4. The method of claim 1, wherein said applying at least one chemical cleaner comprises: rinsing the trays with a quaternary ammonium sanitizer spray, and then submerging the trays in a concentration of up to 300 ppm quaternary ammonium solution for less than 1 minute.

5. The method of claim 1, wherein the desired percent moisture is less than 30% by weight.

6. The method of claim 5, wherein drying the trays is performed with at least one fan and is used to achieve a moisture content ranging from 5 to 15%.

7. The method of claim 1, wherein the dielectric apparatus is a microwave.

8. The method of claim 7, wherein the dielectric apparatus comprises a conveyor belt, a drying tunnel, a controlling system; wherein the conveyor belt is located inside the drying tunnel and the controlling system is attached to the drying tunnel; wherein the drying tunnel has emitters.

9. The method of claim 7, wherein the dielectric apparatus comprised a microwave having at least four emitters that have a power of approximately 10 kW and an output frequency of approximately 2450 MHz.

10. The method of claim 1, wherein the growth tray has a top surface and a bottom surface with a plurality of openings completely through the top surface and the bottom surface.

11. The method of claim 10, wherein the plurality of openings is configured to hold soil and germinate seeds in said soil, the plurality of opening comprising at least three regions:
a top region having a top opening sufficient for sprouting plants to grow through the top surface and sidewalls having a tapered shape to a more narrow transition region;
the transition region having sidewalls that further taper to a more narrow end region; and
an end region having straight sidewalls with a bottom opening for plant roots to grow through the bottom surface and contact an aqueous solution that the growth tray is configured to float on.

12. The method of claim 11, wherein the plurality of openings has an oval shape on the top surface and a round shape on the bottom surface.

13. The method of claim 12, wherein the oval shape on the top surface has as its longest axis a diameter of 18-20 mm.

14. The method of claim 12, wherein the round shape on the bottom surface has a diameter of 8-10 mm.

15. The method of claim 11, wherein the sidewalls of the top region have a taper angle of 2-4 degrees relative to a vertical plane drawn through the center of the openings.

16. The method of claim 15, wherein the top region has a concaved shape bottom leading into the transition region.

17. The method of claim 11, wherein the sidewalls of the transition region have a taper angle of 26-28 degrees relative to a vertical plane drawn through the center of the openings.

18. The method of claim 1, wherein the growth tray is made of expanded polystyrene (EPS).

19. The method of claim 10, wherein the top surface includes a border around the edge that is free of openings, wherein the border has a size at least as wide as the longest axis of the oval shaped openings.

20. The method of claim 19, wherein the border comprises a plurality of tabs that are configured to allow growth trays to be stacked on each other without the bottom surface of a top growth tray touching the top surface of a bottom growth tray stacked on top of it.

21. The method of claim 1, wherein the growth tray is a rectangular shape that is 450-550 mm wide, 700-800 mm long and 50-60 mm thick.

22. The method of claim 1, wherein each growth tray includes from 400-450 openings.

23. The method of claim 22, wherein each opening has a volume ranging from 8-10 ml.
